(19) United States
(12) Patent Application Publication
(10) Pub. No.: US 2024/0248093 A1
(43) Pub. Date: Jul. 25, 2024

(54) ISOLATION AND ANALYSIS OF SINGLE CELLS SECRETING MOLECULES USING HETEROFUNCTIONAL PARTICLES

(71) Applicants: Georgia Tech Research Corporation, Atlanta, GA (US); Emory University, Atlanta, GA (US)

(72) Inventors: Todd Sulchek, Atlanta, GA (US); Ignacio Sanz, Atlanta, GA (US); Frances Eun-Hyung LEE, Atlanta, GA (US); Katily Ramirez, Atlanta, GA (US)

(21) Appl. No.: 18/289,868

(22) PCT Filed: Jun. 3, 2022
(86) PCT No.: PCT/US2022/072754

§ 371 Date: Nov. 7, 2023

Related U.S. Application Data
(60) Provisional application No. 63/196,315, filed on Jun. 3, 2021.

(51) Int. Cl. G01N 33/577; C07K 16/10

(52) U.S. Cl. CPC G01N 33/577; C07K 16/1018

(57) ABSTRACT

An exemplary embodiment of the present disclosure provides a composition comprising a cell capable of secreting one or more molecules, the cell non-covalently attached to a particle, wherein the particle comprises a first linker linking a collector unit, the collector unit capable of binding to the one or more molecules secreted by the cell, optionally, wherein the one or more molecules secreted by the cell are bound to the first unit. The cell is non-covalently bound to the particle through a targeting unit affixed to the particle via a second linker. Also disclosed are methods of isolating cells with one or more particles comprising the same or different collector units and targeting units.

### CROSS-REFERENCE TO RELATED APPLICATIONS

This application claims the benefit of U.S. Provisional Application Ser. No. 63/196,315, filed on 3 Jun. 2021, which is incorporated herein by reference in its entirety as if fully set forth below.

### FEDERALLY SPONSORED RESEARCH STATEMENT

This invention was made with government support under grant/award number DMR-1507238 awarded by the National Science Foundation. The government has certain rights in the invention.

### FIELD OF THE DISCLOSURE

The various embodiments of the present disclosure relate generally to compositions and methods for isolating individual cells, and more particularly to compositions and methods for targeting individuals cells and collecting secreted molecules from the targeted cells.

### BACKGROUND

Emerging pathogens pose tremendous threats globally and to the US. Passive neutralizing antibodies may be helpful to battle threats for viruses such as swine flu, influenza A, or SARS-CoV-2, through the generation of monoclonal antibodies from recovered individuals. Monoclonal antibodies are generated by terminally differentiated B cells known as antibody secreting cells (ASCs), mediators of long-lasting humoral immunity. The surface markers of ASCs are well known, and ASCs can be obtained from blood collected at the peak of the immune response. However, surface expression is downregulated as ASCs differentiate, thus it is difficult to identify antigen specificity by cell surface staining. While intracellular staining with fluorescent antigen can identify antigen-specific ASCs, this method requires cells to be fixed and prevents downstream assays that require viable cells. Specific antigen-specific ASCs can be an extremely small fraction of total ASCs in the blood during infection or vaccination. Therefore, the greatest challenge is the isolation of these rare live antigen-specific ASCs.

Bead-based molecular sensors are convenient platforms to evaluate cytokine, chemokine, and growth factor assays in solution. The bead sensors follow the sandwich assay principle, with the captured molecules immobilized on microbeads, and in the case of Luminex assays, microbeads that possess a unique internal fluorescence label for each measured analyte. Cytokine measurements are performed in a flow cytometer or dedicated Luminex instruments. However, current single-cell protein secretion analysis methods using the bead technology are insufficient in detecting individual cells with high resolution and isolating single cells for future cloning or investigation. Therefore, there is a need for compositions and methods capable of targeting and isolating individual cells secreting ASCs and other secreted molecules.

### BRIEF SUMMARY

The present disclosure relates to compositions and methods for targeting individual cells and collecting secreted molecules from the targeted cells. An exemplary embodiment of the present disclosure provides a composition including a cell capable of secreting one or more molecules and non-covalently attached to a particle. The particle can comprise a first linker linking a first unit. The first unit can be capable of binding to the one or more molecules secreted by the cell. Optionally, the one or more molecules secreted by the cell can be bound to the first unit

In any of the embodiments disclosed herein, the cell can be non-covalently bound to the particle through a second unit. The second unit can be affixed to the particle via a second linker.

In any of the embodiments disclosed herein, the first linker can comprise a silanization binding agent, a carbodiimide binding agent, a carboxylic binding agent, a phosphate binding agent, or combinations thereof.

In any of the embodiments disclosed herein, the second linker comprises a thiol-polymer chain-bioactive molecule complex.

In any of the embodiments disclosed herein, the first unit and second unit each independently comprise a molecule, an antibody, a protein, or combinations thereof.

In any of the embodiments disclosed herein, the first unit can comprise a collector molecule and the second unit can comprise a targeting molecule.

In any of the embodiments disclosed herein, the first unit can comprise a collector antibody and the second unit can comprise a targeting antibody.

In any of the embodiments disclosed herein, the first unit can comprise a collector protein and the second unit can comprise a targeting protein.

In any of the embodiments disclosed herein, the second unit is can be configured to non-covalently attach to a specific cell.

In any of the embodiments disclosed herein, the first unit is can be configured to bind to the one or more molecules secreted by the specific cell.

In any of the embodiments disclosed herein, the composition can be configured for use to detect the one or more molecules secreted from the cell.

In any of the embodiments disclosed herein, the composition can be configured for use to capture the one or more molecules secreted from the cell.

In any of the embodiments disclosed herein, the composition can be configured for use to quantify the one or more molecules secreted from the cell.

In any of the embodiments disclosed herein, the composition can be configured for use to isolate the cell through fluorescence-activated cell sorting (FACS).

In any of the embodiments disclosed herein, the particle further can comprise an outer surface comprising one of hydroxyl or carboxyl functional groups such that the first linker is capable of covalently bonding with the outer surface of the particle.

In any of the embodiments disclosed herein, the particle further can comprise a coating comprising metallic functional groups capable of bonding with the second linker, the coating is positioned on at least a portion of the outer surface of the particle.

In any of the embodiments disclosed herein, the coating can comprise a pattern such that the first unit and the second unit are arranged along the particle in a pattern.

In any of the embodiments disclosed herein, the coating can be positioned on approximately half of the outer surface of the particle, such that a first half of the particle can comprise hydroxyl functional groups and a second half of the particle can comprise metallic functional groups.

In any of the embodiments disclosed herein, the first half of the particle can comprise the first unit and the second half of the particle can comprise the second unit.

In any of the embodiments disclosed herein, the particle can comprise a diameter ranging from about 0.01 μm to about 100 μm.

An exemplary embodiment of the present disclosure provides a composition comprising a particle comprising a cell-binding unit and a molecule-collection unit. The particle can be configured to bind to a specific cell and collect one or more secreted molecules from the specific cell.

In any of the embodiments disclosed herein, the molecule-collection unit can be bound to the particle via a first linker.

In any of the embodiments disclosed herein, the cell-binding unit can be bound to the particle via a second linker.

In any of the embodiments disclosed herein, the first linker can comprise a silanization binding agent, a carboxylic binding agent, a phosphate binding agent, or combinations thereof.

In any of the embodiments disclosed herein, the second linker can comprise a thiol-PEG-biotin complex.

In any of the embodiments disclosed herein, the molecule-collection unit and the cell-binding unit each independently comprise a molecule, an antibody, a protein, or combinations thereof.

In any of the embodiments disclosed herein, the composition can further be configured to detect the one or more molecules secreted from the specific cell.

In any of the embodiments disclosed herein, the composition can further be configured to capture the one or more molecules secreted from the specific cell.

In any of the embodiments disclosed herein, the composition can further be configured to quantify the one or more molecules secreted from the specific cell.

In any of the embodiments disclosed herein, the particle can further comprise an outer surface comprising one of a hydroxyl or a carboxyl functional groups such that the first linker can be capable of covalently bonding with the outer surface of the particle.

In any of the embodiments disclosed herein, the particle can further comprise a coating comprising metallic functional groups capable of bonding with the second linker, the coating is positioned on at least a portion of the outer surface of the particle.

In any of the embodiments disclosed herein, the coating can be positioned on approximately half of the outer surface of the particle, such that a first half of the particle can comprise hydroxyl functional groups and a second half of the particle can comprise metallic functional groups.

An exemplary embodiment of the present disclosure provides a method of isolating and expanding a cell, the method can comprise contacting the cell with a particle, binding the cell, capturing one or more molecules secreted from the cell, and sorting the cell bound to the particle. The particle can comprise a cell-binding unit and a molecule-collection unit. Binding the cell can be via the cell-binding unit of the particle. Capturing one or more molecules secreted from the cell can be via the molecule-collection unit of the particle. Sorting the cell bound to the particle can optionally be by one or more detecting units specific to the one or more molecules secreted from the cell.

In any of the embodiments disclosed herein, the method can further comprise releasing the cell from the particle.

In any of the embodiments disclosed herein, the method can further comprise expanding the cell.

In any of the embodiments disclosed herein, the method can further comprise detecting a quantity of the one or more molecules secreted from the cell, optionally using the one or more detecting units specific to the one or more molecules secreted from the cell.

In any of the embodiments disclosed herein, the method can further comprise separating a low-secretion cell from a high-secretion cell based on the quantity of the one or more molecules secreted from the respective cell.

An exemplary embodiment of the present disclosure provides a method of isolating and expanding a cell. The method can comprise contacting the cell with a first particle and a second particle, binding the cell at a first position, binding the cell at a second position, capturing one or more first secreted molecules from the cell, capturing one or more first secreted molecules from the cell, capturing one or more second secreted molecules from the cell, sorting the cell based on a type of first secreted molecule, and sorting the cell based on a type of second secreted molecule. Each respective particle can comprise a cell-binding unit and a molecule-collection unit. The cell-binding unit and the molecule-collection unit on the respective particle can comprise at least one of a molecule, an antibody, or a protein. The cell-binding unit and the molecule-collection unit on the first particle can be different than the cell-binding unit and the molecule-collection unit on the second particle. Binding the cell at a first position can be via the cell-binding unit of the first particle. Binding the cell at a second position can be via the cell-binding unit of the second particle. Capturing one or more first secreted molecules from the cell can be via the molecule-collection unit of the first particle, Capturing one or more second secreted molecules from the cell can be via the molecule-collection unit of the second particle. Sorting the cell based on a type of first secreted molecule can be achieved by one or more first detecting units specific to the one or more first secreted molecules from the cell based on a type of first secreted molecule. Sorting the cell based on a type of second secreted molecule can be achieved by one or more second detecting units specific to the one or more second secreted molecules from the cell based on a type of second secreted molecule.

In any of the embodiments disclosed herein, the method can further comprise sorting the cell based on a quantity of the first secreted molecule and second secreted molecule from the cell using the one or more first detecting units and one or more second detecting units.

In any of the embodiments disclosed herein, the method can further comprise releasing the cell from the first and second particles.

In any of the embodiments disclosed herein, the method can further comprise expanding the cell.

An exemplary embodiment of the present disclosure provides a method of producing secreted antibodies. The method can comprise contacting a cell with a particle, capturing one or more secreted molecules from the cell, and sorting the cell bound to the particle. The particle can comprise a cell-binding unit and a molecule-collection unit. Capturing one or more secreted molecules from the cell can be via the molecule-collection unit of the particle. Sorting the cell bound to the particle can optionally be achieved via the one or more detecting units specific to the one or more molecules secreted from the cell.

In any of the embodiments disclosed herein, the method can further comprise identifying the cell based on a type of the one or more secreted molecules.

In any of the embodiments disclosed herein, the method can further comprise isolating the cell based on the type of the one or more secreted molecules.

In any of the embodiments disclosed herein, the method can further comprise expanding the cell based on the type of the one or more secreted molecules, such that the cell is capable of producing a specific secreted antibody.

In any of the embodiments disclosed herein, the method can further comprise identifying the cell as a high secretion cell or a low secretion cell based on a quantity of the one or more secreted molecules.

In any of the embodiments disclosed herein, the method can further comprise simultaneously identifying the cell based on a marker on the cell bound to the particle via the molecule-collection unit of the particle.

In any of the embodiments disclosed herein, the method can further comprise isolating the cell based on the quantity of the one or more secreted molecules.

In any of the embodiments disclosed herein, the method can further comprise expanding the high secretion cell, such that the high secretion cell is capable of producing a specific secreted antibody.

In any of the embodiments disclosed herein, the method can further comprise expanding the low secretion cell, such that the low secretion cell is capable of producing a specific secreted antibody.

In any of the embodiments disclosed herein, the method can further comprise generating monoclonal antibodies from the one or more secreted molecules from the cell.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a schematic illustration of an example composition for isolating cells, collecting secreted molecules, and/or producing secreted antibodies, in accordance with an exemplary embodiment of the present invention.

FIGS. 1B and 1C provide schematic illustrations of example particles having a coating, a collector molecule, and a targeting molecule, in accordance with an exemplary embodiment of the present invention.

FIG. 2A provides a schematic illustration of an example mechanism of an isolation and expansion assay using the composition, in accordance with an exemplary embodiment of the present invention.

FIG. 2B provides a schematic illustration of an example particle having the coating on approximately half of the outer surface, in accordance with an exemplary embodiment of the present invention.

FIG. 3A provides an example schematic of an experiment with TIB147 and BCL6 to identify anti-concanavalin A and anti BCL6, in accordance with an exemplary embodiment of the present invention.

FIG. 3B provides example parameters for the contingency table and equations used for sensitivity, specificity, and accuracy, in accordance with an exemplary embodiment of the present invention.

FIGS. 3C and 3D provide example gating strategies to isolate high and low molecule-secreting cells, in accordance with an exemplary embodiment of the present invention.

FIGS. 4A through 4D provide composition functionality via flow cytometry plots and confocal images of example compositions with particle control (FIG. 4A), with gold (FIG. 4B), with silica (FIG. 4C), and with both silica and gold (FIG. 4D), in accordance with an exemplary embodiment of the present invention.

FIGS. 5A through 5E provide example comparison between patterns of coatings on the outer surface of particles, in accordance with an exemplary embodiment of the present invention.

FIG. 6 provides a schematic illustration of a process of isolating one or more secreted molecules using the composition, in accordance with an exemplary embodiment of the present invention.

FIG. 7 provides a diagram illustrating an example process of expanding isolated cells using the particle, in accordance with an exemplary embodiment of the present invention.

FIG. 8 provides a diagram illustrating an example process of expanding isolated cells using the two different particles, in accordance with an exemplary embodiment of the present invention.

FIG. 9A provides a plot showing CD 107a fold increase for various particles, in accordance with an exemplary embodiment of the present invention.

FIG. 9B provides a plot showing mean fluorescence intensity of FITC anti-CD107a per cell for various particles, in accordance with an exemplary embodiment of the present invention.

FIG. 10A provides a plot of IL-2 concentration for various particles and percentage increase of IL-2 concentration between unsorted cells and isolated high and low IL-2 secreting cells, in accordance with an exemplary embodiment of the present invention.

FIG. 10B provides confocal images and MFI of the detection antibody of an isolated high secreting cells and a low secreting cells, in accordance with an exemplary embodiment of the present invention.

FIGS. 11A and 11B provide plots of IL-2 concentration for high and low secretors for various particle coating types including Janus particles (FIG. 11A), mixed particles (FIG. 11B), in accordance with an exemplary embodiment of the present invention.

FIG. 11C provides a plot of MFI of FITC anti-IL2 per particle for either mixed particles or Janus particles, in accordance with an exemplary embodiment of the present invention.

FIGS. 12A and 12B provide plots of inheritability of IL-2 secretion in Jurkat cells, in accordance with an exemplary embodiment of the present invention.

FIG. 13 provides a plot of concentration of IL-2 versus concentration of VEGF of unsorted, high secretors, and low secretors isolated by the particles, in accordance with an exemplary embodiment of the present invention.

FIG. 14A provides a plot of concentration of IgG from high secretors and low secretors isolated by the particles, in accordance with an exemplary embodiment of the present invention.

FIGS. 14B and 14C provides plots of number of particles per cell after trypsinization for particles added before, during, and after cell trypsinization, in accordance with an exemplary embodiment of the present invention.

FIGS. 15A through 15D provide plots of concentration of VEGF from high secretors and low secretors isolated by various particles including Janus particles after 4 days of incubation (FIG. 15A), mixed particles after 4 days of incubation (FIG. 15B), and Janus particles after 8 days of incubation (FIG. 15C), mixed particles after 8 days of incubation (FIG. 15D), in accordance with an exemplary embodiment of the present invention.

FIGS. 16A and 16B provide plots of proliferation index of isolated high and low VEGF secreting cells isolated with Janus particles (FIG. 16A) and mixed particles (FIG. 16B) after 4 days of incubation, in accordance with an exemplary embodiment of the present invention.

FIG. 16C provides a plot of proliferation index of cells isolated with Janus particles and mixed particles after 8 days of incubation, in accordance with an exemplary embodiment of the present invention.

FIGS. 17A and 17B provide plots of percentage of viable cells indicating no toxic effect of particles, in accordance with an exemplary embodiment of the present invention.

FIGS. 18A through 18E provide plots demonstrating optimal conditions for efficient cells targeting with labeled antibody maximizing antigen intensity per particle, in accordance with an exemplary embodiment of the present invention.

FIG. 19A provides a plot of percentage of cells targeted before and after sorting of particles based on cell type, in accordance with an exemplary embodiment of the present invention.

FIG. 19B is a confocal image of Jurkat and blue cell tracker, TIB147, targeted by a 4 m mixed particle with FIC Anti-CD44, in accordance with an exemplary embodiment of the present invention.

FIG. 19C is a plot of particles per cell after sorting the particle for various cell types, in accordance with an exemplary embodiment of the present invention.

FIG. 20 provides a plot of percentage of sensitivity, specificity, and accuracy for either Janus particles or mixed particles of various sizes, in accordance with an exemplary embodiment of the present invention.

FIGS. 21A and 21B provide correlation of intensity of H1 and H3 tetramer and concentration of H1IV and H3IV antibody using particles, in accordance with an exemplary embodiment of the present invention.

FIG. 22 provides an example ELISPOT result for a sample patient “2600” for day 6 post vaccination, in accordance with an exemplary embodiment of the present invention.

FIG. 23 is a flow diagram outlining steps outlining a method of isolating cells with a single particle, in accordance with an exemplary embodiment of the present invention.

FIG. 24 is a flow diagram of outlining a method of isolating cells with more than one particle, in accordance with an exemplary embodiment of the present invention.

FIG. 25 is a flow diagram outlining steps outlining a method of producing secreted cells, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 710% to 99%.

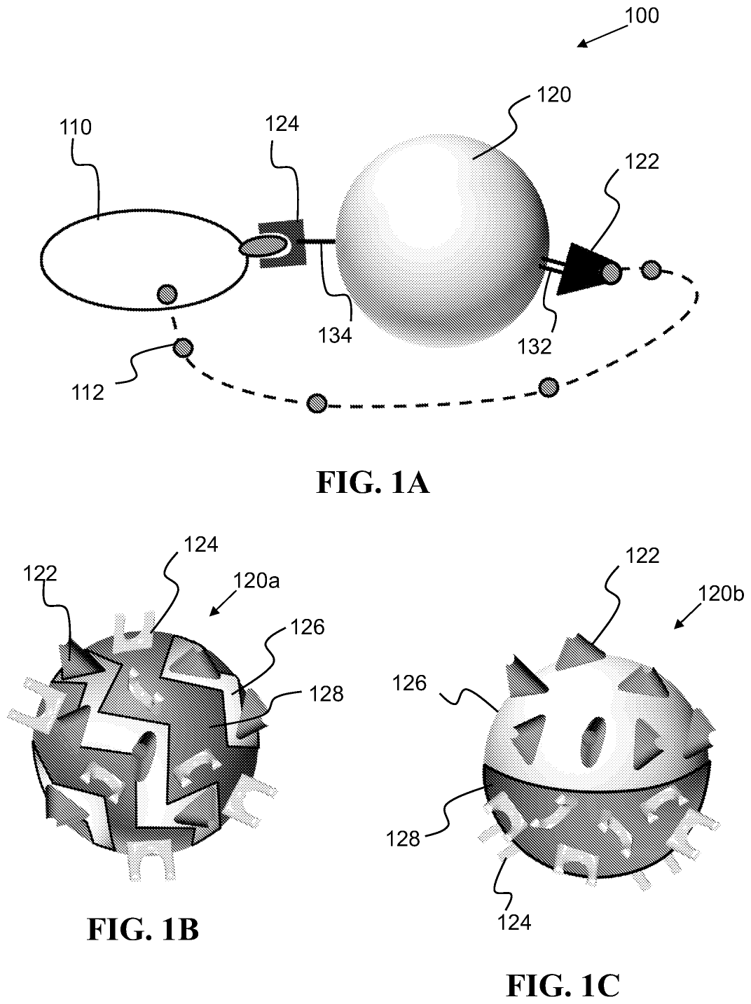

As shown in FIG. 1A, an exemplary embodiment of the present invention provides a composition 100 for isolating cells, collecting secreted molecules, and/or producing secreted antibodies. Composition 100 can include a cell 110 that secretes one or more molecules 112. Cell 110 can be bound to a particle 120 having a first unit 122 capable of collecting the one or more molecules 112 secreted from cell 110. Cell 110 can be bound to particle 120 via a second unit 124 of particle 120, where second unit 124 is capable of targeting a specific cell, as described in more detail below. First unit 122 can also function as a collecting unit and can be configured to collect specific secreted molecules from the cell based on the collector molecule attached to particle 120 via a first linker 132. Similarly, second unit 124 can function as a targeting unit and can be configured to reversibly bind to a specific marker on a specific cell based on the targeting molecule attached to particle 120 via a second linker 134.

Particle 120 can include metal oxide particles having hydroxyl functional groups on the surface, such as, for example, silicon dioxide (silica), tin oxide, aluminum oxide, magnesium oxide, zirconium oxide, zinc oxide, copper oxide, silver oxide, titanium dioxide, iron oxide, cerium oxide, and the like. Alternatively, or in addition thereto, particle 120 can include particles having surface carboxyl functional groups such as, for example, polystyrene. polybutyl acrylate, polymethacrylic acid, polyvinyl, and the like. For particles having surface carboxyl groups, crosslinkers can be used to bind the collecting unit to the particle. Crosslinkers can include, for instance, 1-Ethyl-3-[3-dimethylaminopropyl]carbodiimide hydrochloride (EDC), N-hydroxysuccinimide (NHS) and the like.

In some embodiments, particle 120 can have average particle sizes (e.g., average particle diameter) ranging from about 0.01 μm to about 100 μm (e.g., from about 0.05 μm to about 0.1 μm, about 0.15 μm to about 0.2 μm, about 0.25 μm to about 0.3 μm, about 0.35 μm to about 0.4 μm, about 0.45 μm to about 0.5 μm, about 0.55 μm to about 0.6 am, about 0.65 m to about 0.7 μm, about 0.75 μm to about 0.8 μm, about 0.85 μm to about 0.9 am, about 0.95 μm to about 1 μm, about 1 μm to about 2 μm, about 2 μm to about 3 am, about 3 μm to about 4 μm, about 4 μm to about 5 μm, about 5 μm to about 6 am, about 6 μm to about 7 am, about 7 μm to about 8 μm, about 8 μm to about 9 am, about 9 μm to about 10 am, about 10 m to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, about 90 μm to about 100 am, or any value between, e.g., 0.72 μm or 51 μm).

In some embodiments, particle 120 can be used to activate and/or promote production of secreted molecules 112 from a population of cells 110. Simultaneously or sequentially, particle 120 can be used to collect secreted molecules 112 produced from cell 110. The secreted molecules can include cytokines, chemokines, antibodies, growth factors, exosomes, and the like. In some examples, the secreted cytokines can be TNF-α, CXCL8 (formerly IL-18), IL-23, IP-10, MIP-1α, MCP-1, G-CSF, GM-CSF, Interferons type I, II, III, IL-2, IL-3, IL-4, IL-5, IL-6, IL-7, IL-8, IL-9, IL-10, IL-12, IL-21, and many other secreted cytokines and the like. Secreted factors that may be released to influence somatic cell reprogramming may include Sostdc1, Glb112, Fetub, Dpp4, Gdf3, Trh, and Tdgf1. Secreted factors may also be involved in endocrine signaling, for example erythropoietin, glucagon, insulin, estrogen, progesterone, thyroid hormone, epinephrine, testosterone, melatonin, growth hormone releasing hormone, thyrotropin releasing hormone, humoral factors, and the like. Alternatively, or in addition thereto, cells can be induced to secrete antibodies such as IgG, IgA, IgD, IgE, IgM, synthetic antibodies, and the like. Alternatively, or in addition still, cells can be induced to secrete growth factors such as, for example, PDGF, VEGF, EGF, FGF, HGF, NGF, and the like. Alternatively, or in addition thereto, cells can be induced to secrete exosomes including, for instance, insulin receptor substrate, VEGF, IgM, PDGF, PEDF, and the like.

In some embodiments, collector molecule 122 can be a molecule, antigen, antibody, or protein specific epitope of the one or more secreted molecules from the cell. In some embodiments, collector molecule 122 is capable of specifically binding to the one or more secreted molecules 112. For example, a collector molecule can include anti-IL2 in order to bind to and collect secreted IL-2. In another example, a collector molecule can include anti-VEGF in order to bind to and collect secreted VEGF. Other example collector molecules can include but are not limited to hormones, signaling molecules, reprogramming factors, innate immune products such as complement proteins, and the like. For instance, protein G in order to collect IgG antibodies, Protein A for IgA antibodies.

In any of the embodiments described herein, particle 120 can be coated with targeting molecules 124 to bind to and induce secretion of the molecules 112 from cells 110. In particular, targeting molecule 124 can be tailored to target and bind a specific cell. For instance, to target a B lymphocyte, targeting molecule 124 can include a specific antigen and/or peptide capable of targeting a single antibody on a B lymphocyte. Example B cell antibodies can include, for instance IgM, CD19, CD25, CD30, CD38, IgG, IL-6, CD138, Notch2, CD38, CD27, CD20, B220, and the like.

Additionally, or alternatively thereto, to target a T lymphocyte, targeting molecule 124 can include a specific antigen and/or peptide that is capable of binding to specific T cell receptor. Example T cell receptors can include, for instance, CD3, CD4, CD5, CD7, CD8, CD27, CD28, CD45, CD45RA, CD62L, CD69, CD103, CCR7, CXCR3, and the like. As a non-limiting example, a targeting molecule can include anti-CD3 and/or anti-CD28 such that the particle is capable of binding to a CD3 receptor or a CD28 receptor on a T cell. Additional examples include CD4, CD5, CD7, CD8, CD27, CD45, CD45RA, CD62L, CD69, CD103, CCR7, CXCR3, and the like.

In an embodiment, the targeting molecule can be a bi-specific antibody capable of binding to multiple antigens on the surface of the cells 110.

FIGS. 1B and 1C provide schematic illustrations of non-limiting example particles 120a, 120b that can be used in composition 100. Particle 120 can include an outer surface 126 that can be functionalized to bind varying targeting units and collecting units. In some examples, particle 120a, 120b can have a coating 128 positioned proximal at least a portion of outer surface 126. For instance, as shown in FIG. 1B, coating 128 can positioned in an arrangement such as stripes, checkers, zig-zag, or random such that the collector units 122 and targeting units 126 are mixed along the particle, sometimes referred to as “mixed particles.” Alternatively, or in addition thereto, coating 128 can be positioned along approximately half of the particle surface 126, forming two hemispheres, such that the collector units 122 are positioned on a first half of the particle surface 126 and the targeting units 124 are positioned on a second half of the particle surface 126, as depicted in FIG. 1C, and sometimes referred to herein as “Janus particles.” Although not shown in FIGS. 1B and 1C, the collector units 122 and targeting units 124 can be bound to particle 120 via a first linker 132 and a second linker 134, respectively.

In some embodiments, coating 128 can generate metallic functional groups on outer surface 126 of particle 120. In general, metal-based materials that can form oxidative metal surfaces such that a metal-sulfur bond can form via a chemisorbed interaction can be used to form the thiol-polymer chain-bioactive molecule linker between particle 120 and second linker 134. Coatings can include, for example, gold, silver, titanium, vanadium, chromium, iron, cobalt, copper, zinc, zirconium, niobium, molybdenum, ruthenium, rhodium, palladium, silver, cadmium, hafnium, tantalum, tungsten, osmium, iridium, platinum, and combinations thereof.

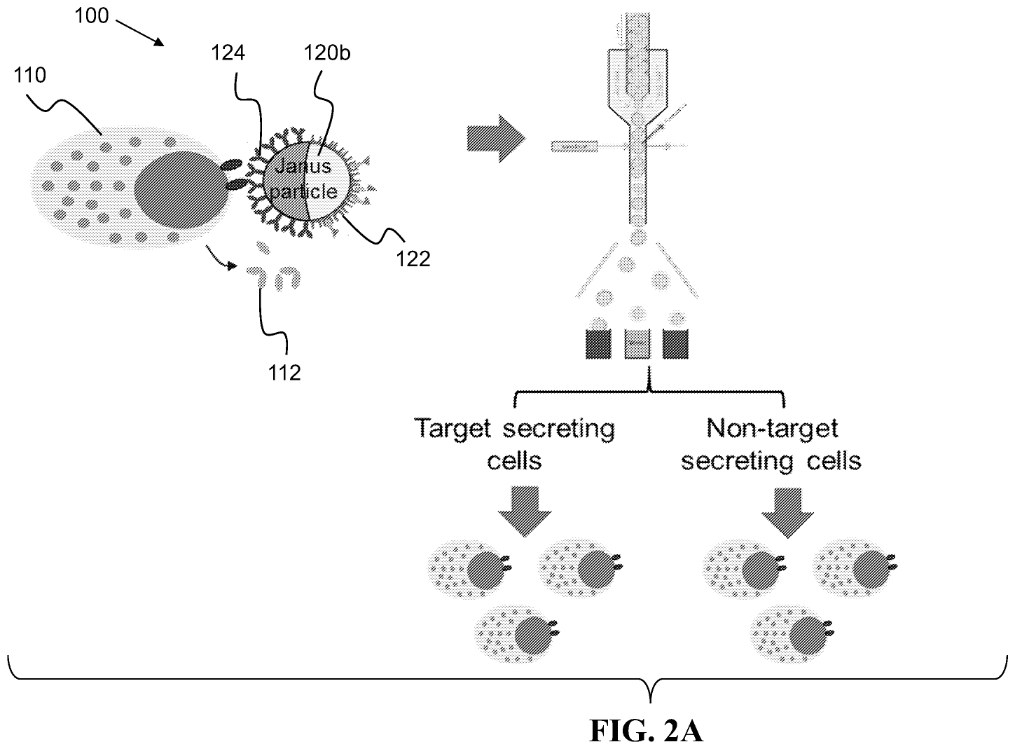

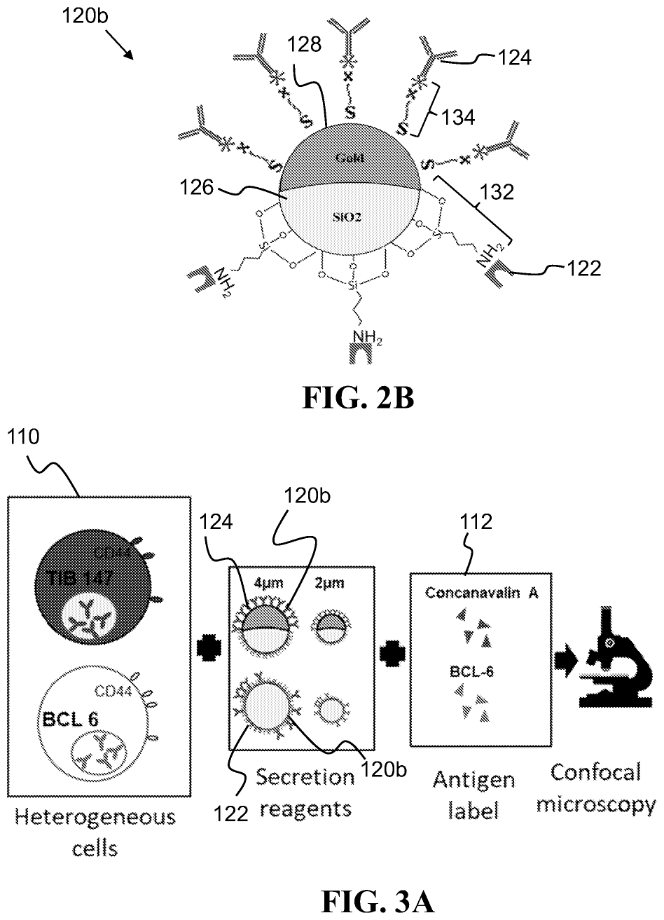

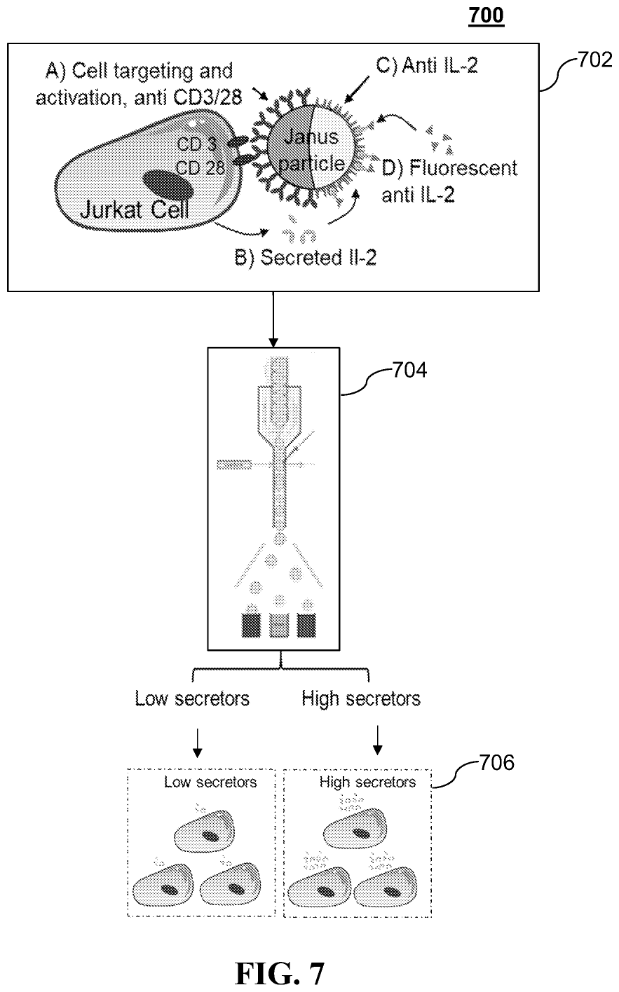

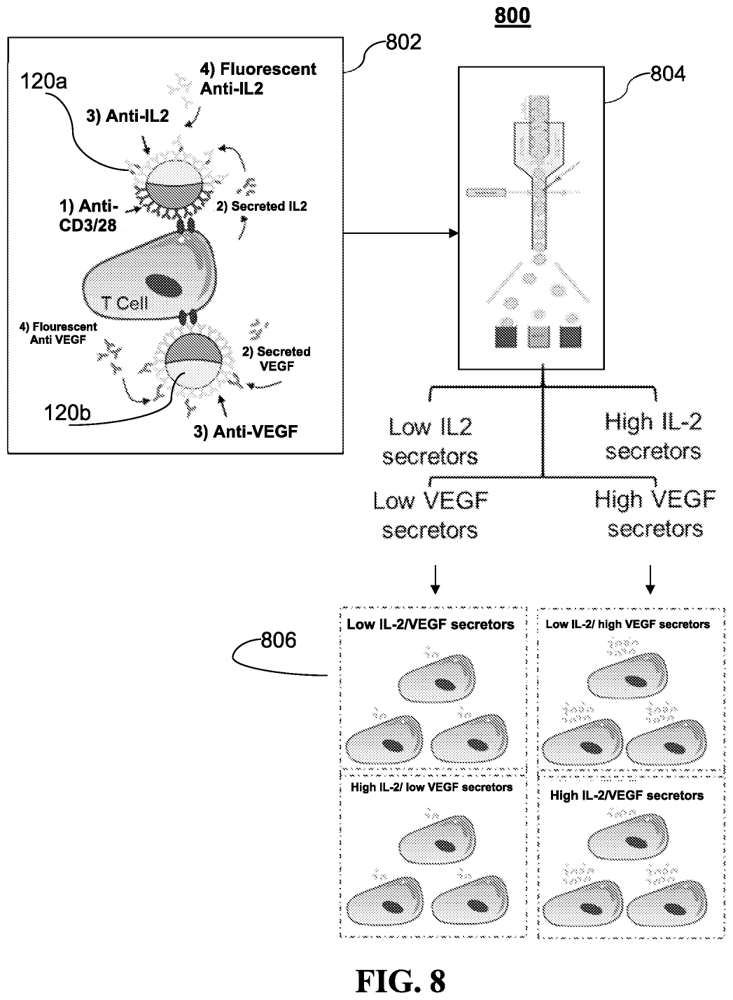

FIG. 2A depicts a schematic illustrating an example mechanism of an isolation and expansion assay using the composition 100 described herein. As shown, composition 100 can include the cell 110 bound to the particle 120b via the targeting unit 124. When the particle 120 binds to the cell, the cell can be activated to secrete one or more molecules 112. Because particle 120 is in close proximity to cell 110 bound to the particle, the collecting unit 122 of particle 120b is capable of binding the one or more molecules 112 secreted from cell 110. After composition 100 is formed, the particle 120b can be sorted using a suitable cell-sorting technique, such as, for example, fluorescence-activated cell sorting (FACS), microfluidics enabled cell sorting, size based sorting, mass sorting, magnetic sorting, closed-loop GMP compliant sorting, and the like. Particles 120b can be sorted for those that are bound to a target cell of interest from those that are non-targeted cells, as depicted in FIG. 2A, or can further be sorted based on the quantity of secreted molecules, as depicted in FIGS. 7 and 8 and described in more detail below.

FIG. 2B is a schematic illustration of an example particle 120b, illustrating example linkers 132, 134. As shown, particle 120b can have coating 128 on approximately half of outer surface 126 such that collector unit 122 is positioned on a first hemisphere of particle 120b and targeting unit 124 is positioned on a second hemisphere of particle 120b. First linker 132 between particle 120 and collector molecule 122 can include a binding agent that can covalently bond with hydroxyl functional groups on outer surface 126 of uncoated portions of particle 120. Suitable binding agents can include, for example, silylanization binding agents (e.g., (3-aminopropyl)triethoxysilane, (3-aminopropyl)trimethoxysilane, hexamethyldisilazane, trimethoxy(octadecyl)silane, 3-mercaptopropyltrimethoxisilane, 2-aminophenyldisulfide, 3-glycidoxypropryltrimethoxysilane, and the like), carbodiimide binding agents (e.g., 1-Ethyl-3-(3-dimethylaminopropyl)carbodiimide, dicyclohexylcarbodiimide, N,N′-Diisopropylcarbodiimide, 1-cyclohexyl-(2-morpholinoethyl)carbodiimide metho-p-toluene sulfonate and the like), carboxylic binding agents (e.g., glutaraldehyde, and the like), phosphate binding agents, or combinations thereof. In some embodiments, a first linker 132 with an amine functional group can be used to bind to a carboxyl in the collector molecule 122 that is a molecule, a protein, or an antibody. As would be appreciated by one of skill in the art, first linker 132 can vary in length between the particle and the collecting unit. In some examples, a longer first linker leads to more degrees of freedom by which the collecting unit and cell-secreted molecules can bind. In addition, different first linkers 132 may have varying binding affinity between the particle and the collecting unit that may be adjusted for improved sensitivity, specificity, and accuracy.

Referring back to FIG. 2B, second linker 134 between particle 120 and targeting molecule 124 can include a suitable linking complex that can bind with metal-based functional groups on coating 128 of coated portions of particle 120. Suitable linking complexes can include any suitable complex that has a free thiol group on one end, a polymer chain, and a bioactive small molecule on a second end. Suitable linking complexes can include, for example, thiol-PEG-biotin complexes, thiol-PEG-chitosan complexes, thiol-PEG-cellulose complexes, thiol-PEG-methoxyl complexes, thiol-PEG-carboxylic complexes, thiol-PEG-lipoic acid complexes, thiol-PEG-fluorescein complexes, thiol-PEG-methoxyl silane complexes, thiol-PEG-succinimidyl ester complexes, thiol-PEG-maleimide complexes, and the like. As would be appreciated by one of skill in the art, second linker 134 can vary in length between the particle and the targeting unit by adjusting the polymer chain to a different length of a different type of polymer (e.g., polypropylene glycol and the like). In some examples, a longer second linker leads to more degrees of freedom by which the targeting unit and cell can bind. In addition, different second linkers 134 may have varying binding affinity between the particle and the targeting unit that may be adjusted for improved sensitivity, specificity, and accuracy.

In a particular example, FIG. 2B shows a Janus particle conjugation with streptavidin conjugated anti-CD44 on gold-coated hemisphere by thiol-PEG-biotin and Protein G on silica hemisphere by means of a silylanization binding agent with amino(propyl)triethoxysilane (APTES).

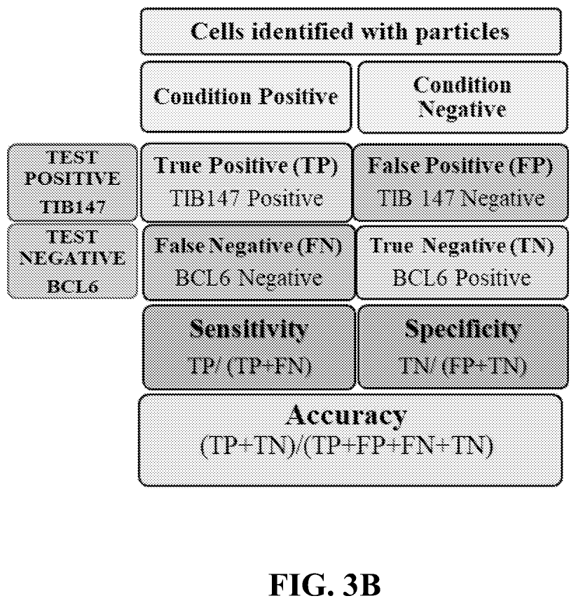

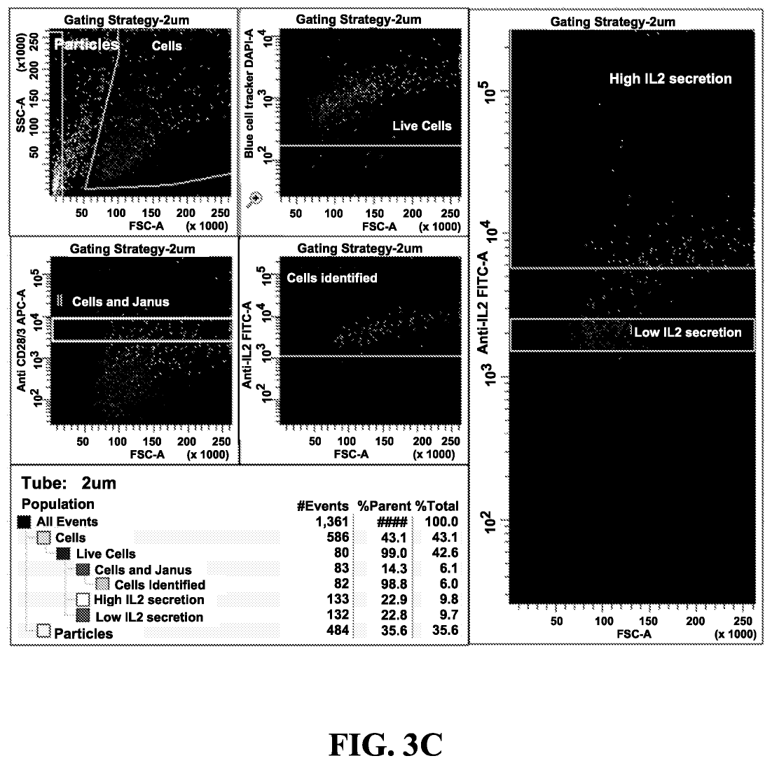

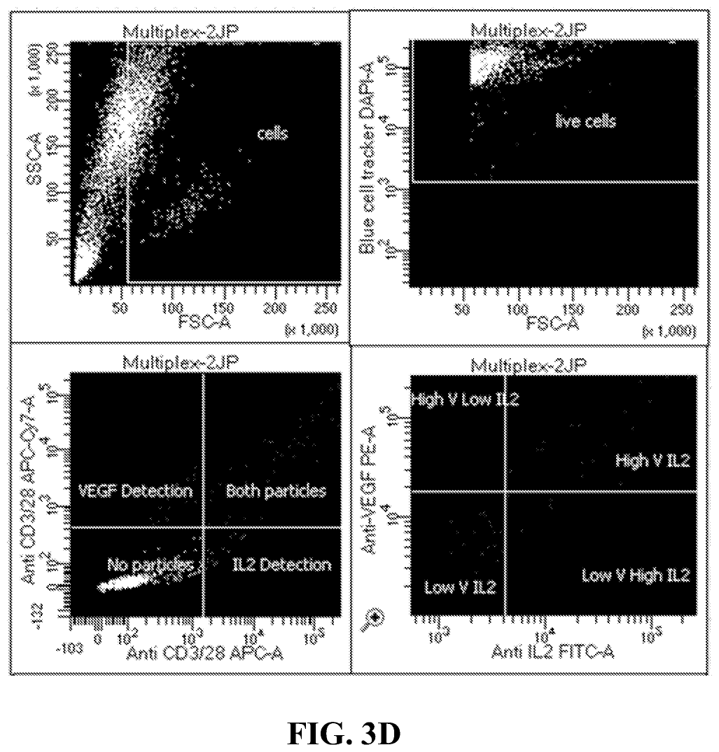

FIG. 3A provides an example schematic of an experiment with cells 110 (TIB147 and BCL6) identified, via confocal microscopy, by introducing particles 120a, 120b for binding to cells 110 with respective targeting units 124, and further for collecting one or more molecules 112 (concanavalin A and BCL6) with antigen labels specific to the one or more molecules (anti-concanavalin A and anti BCL6).

FIG. 3B provides example parameters for an experiment with TIB147 and BCL6 to identify anti-concanavalin A and anti-BCL6 as described in FIG. 3A. The contingency table also provides example equations used for sensitivity, specificity, and accuracy of identifying cells bound to particles and secreting concanavalin A and BCL6.

FIGS. 3C and 3D provide example gating strategies to isolate high and low IL-2 secreting Jurkat cells. For instance, a first gate can include cells based on FSC and SCS. A second gate can include cells with blue cell tracker. A third gate can include cells with APC, representing the targeting antibody on the particles. A fourth gate can include cells with different intensity of FITC, representing the detection antibody, and high and low IL-2 secreting cells. In one example, as shown in FIG. 3D, a gating strategy to isolate high and low IL-2 and VEGF secreting Jurkat cells can include up to four different steps. A first gate can include cells based on FSC and SCS. A second gate can include cells with blue cell tracker, representing viable cells. A third gate can include cells with APC and APC cy 7, representing the targeting antibody on both particles. A fourth gate can include cells with different intensity of FITC and PE, representing the detection antibody for IL-2 and VEGF, respectively.

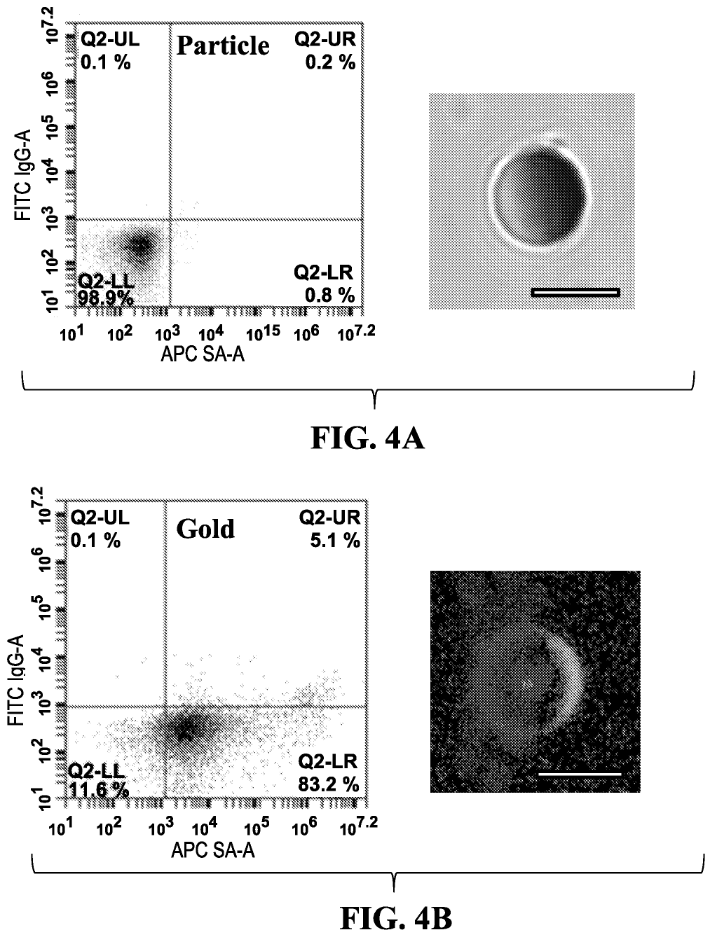

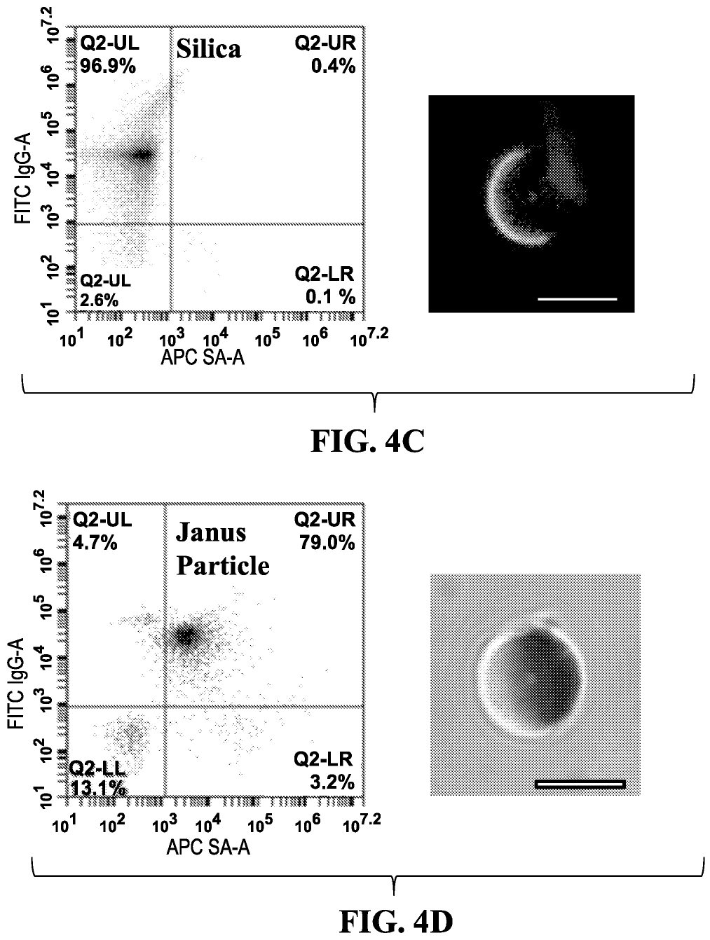

FIGS. 4A through 4D provide composition functionality via flow cytometry plots and confocal images of example compositions with a control particle in FIG. 4A, a particle with a coating comprising gold in FIG. 4B, a particle comprising silica in FIG. 4C, and a particle with both silica and gold in FIG. 4D. Specifically, FIGS. 4A-4D demonstrate functionality of a particle having various outer surface functionalities from the silica and gold coating that enable capturing specific cells with the targeting unit and collecting secreted molecules with the collecting unit. For instance, as a non-limiting example, 4 μm silica Janus particles shown in FIG. 4D can be functionalized such that APC-streptavidin (APC SA-A) can be conjugated on a gold-coated hemisphere and FITC antibody bound to Protein G (FITC IgG-A) can be conjugated on a silica functionalized hemisphere.

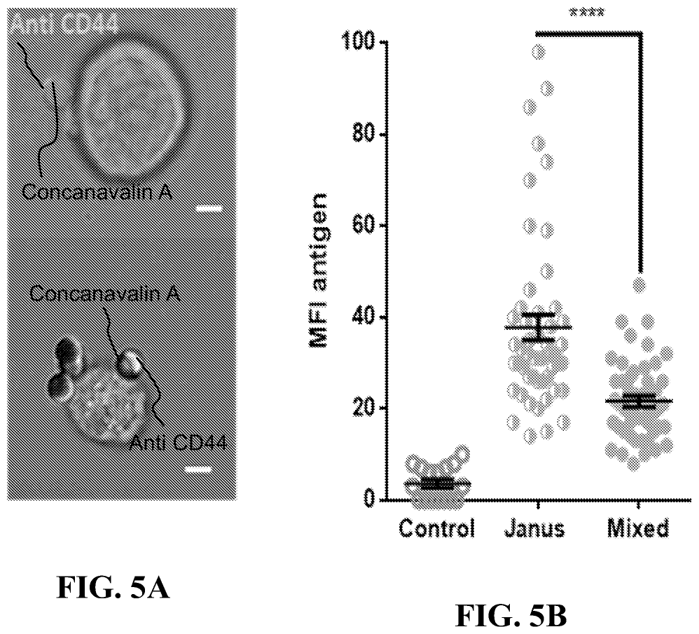

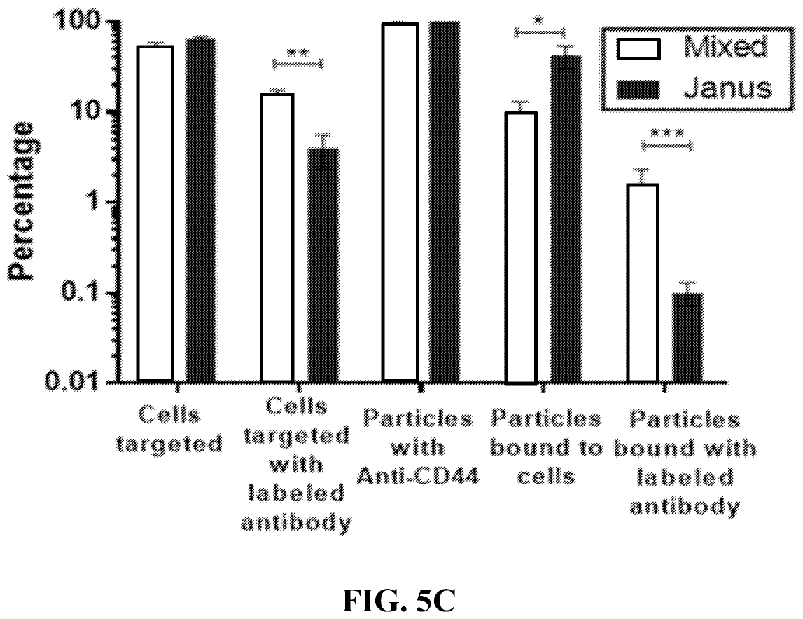

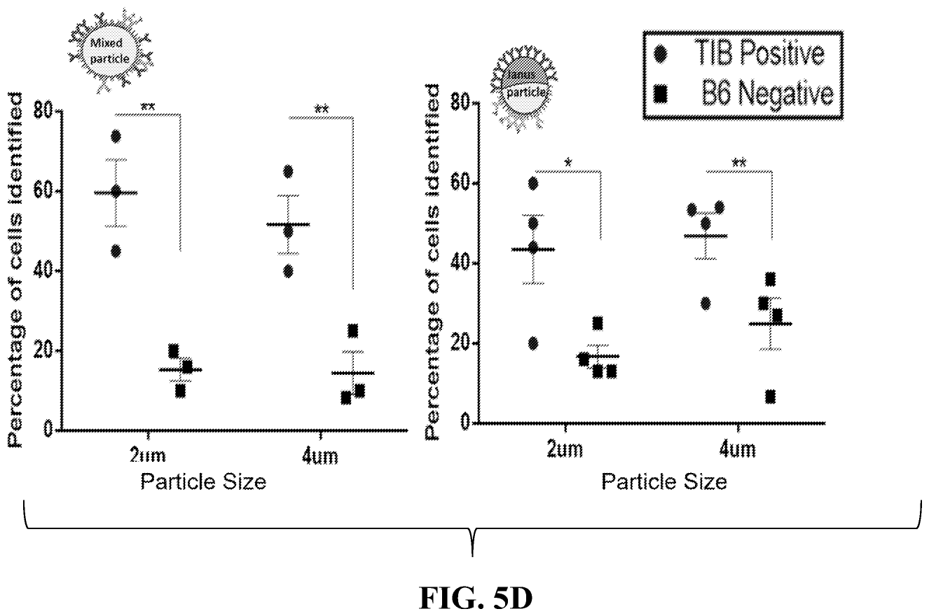

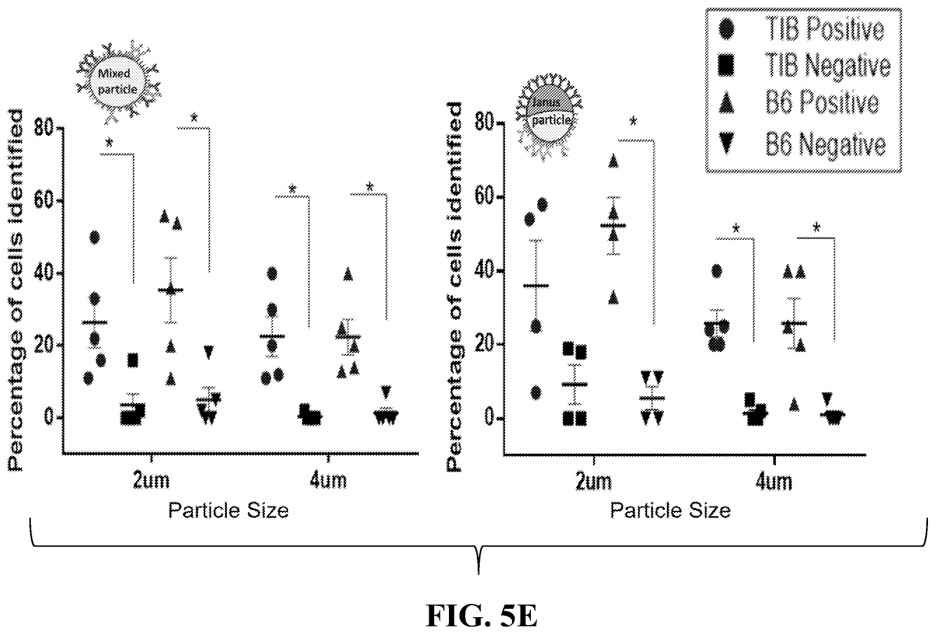

FIGS. 5A through 5E provide comparison between patterns of coatings 128 on outer surface 126 of particles 120, specifically between mixed particles and Janus particles compared to a control particle with no functionalization. Although not depicted, additional patterns for functionalizing particles are contemplated and can be accomplished through etching of the outer surface to remove metal-containing coatings and reveal hydroxyl group on the particle.

FIG. 5A is confocal images of about 4 μm silica particle having a mixed pattern (top) and a Janus pattern (bottom), each particle targeting TIB147 hybridomas through anti-CD44, collecting anti-Concanavalin A and antibody labeling using fluorescent Concanavalin A, where the scale bar is 4 μm. FIG. 5B is a plot of labeled antibody of the Janus particles and mixed particles selected by attachment to cells, where **** equal to p<0.0001. FIG. 5C is a bar graph demonstrating efficiency of targeting and collection of 4 μm Janus particles and mixed particles, where *, **, and *** equal p<0.05, p<0.01, and p<0.001, respectively. FIG. 5D shows percentage of cells identified in a complex mixture of cells for identification of TIB147 anti-concanavalin A secreting cells (n=3 for mixed and n=4 for Janus particles). FIG. 5E shows percentage of cells identified in a complex mixture of cells for identification of both TIB147 anti-concanavalin A and B6 anti-BCL6 secreting cells in a mixture of B6 and TIB147 cells using mixed particles (left) and Janus (right) particles, where * and ** equal to p<0.05 and 0.001 respectively (n=5 for mixed and n=4 for Janus particles).

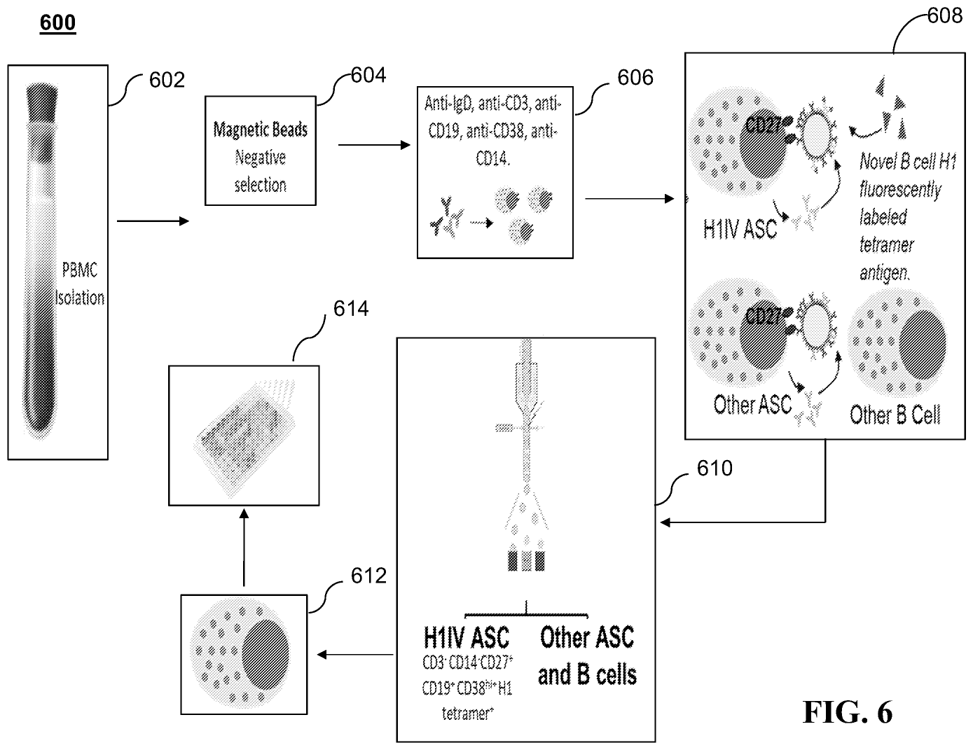

FIG. 6 provides a schematic illustration of an example process 600 of isolating one or more secreted molecules 112 using composition 100. As a non-limiting example, composition 100 can be used for isolation of anti-H1HA antibody secreting cells using steps of collecting 602 PBMC, enriching 604 B cells, staining 606 cell surfaces, capturing 608 secreted antibodies from targeted cells, sorting and isolating 610 single cells secreting specific antibody secreted cells (ASCs), incubating 612 sorted cells, and validating 614 antibody secretion via ELISA. In the particular example shown in FIG. 6, steps 608 through 612 show how the mixed particle can bind to ASC via anti CD27 and the secreted antibodies can be collected via Protein G and identify H1HA specific ASC using a fluorescent labeled antigen and validation using anti-H1HA antibody secretion and validation with ELISA.

FIG. 7 provides a diagram illustrating an example process 700 of expanding isolated cells using composition 100. As a non-limiting example, composition 100 can bind to a Jurkat cell through anti-CD3 and anti-CD28 such that the Jurkat cell is activated to secrete IL-2 and composition 100 can collect IL-2, as shown in more detail in the schematic of step 702. Fluorescent detection of collected IL-2 can be used for isolating and sorting high and low IL-2 secreting cells via FACS, depicted in the schematic of step 704. Process 700 can end at cell expansion of high and low IL-2 secretor cells as shown in step 706 or can continue further for validation of high and low IL-2 secretors via ELISA or any other suitable validation technique. In some embodiments, protein production can be more favorable to isolate high secretor cells. In certain examples, low secretor cells can be for cell analysis, like for example stimulation performance analysis, cell exhaustion, isolation of cells will low level of Insulin receptor substrate for diabetes.

In some embodiments, detecting units can be fluorescently-labelled molecules such as antibodies, tetramers, proteins, viruses, cytokines, epitopes, antigens, binding partners, and the like. Additionally, detecting of a quantity or a type of molecules secreted from a cell can be conducted using any suitable cell detection method such as, for example, FACS, optical microscopy, plate reader, enzymatic reactions such as ELISA, and the like.

FIG. 8 provides a diagram illustrating an example process 800 of expanding isolated cells using two different particles 120a, 120b. As a non-limiting example, a first particle 120a can bind to a Jurkat cell through anti-CD3 and a second particle 120b can bind to the Jurkat cell through anti-CD28 such that the Jurkat cell is activated to secrete IL-2 and VEGF. First particle 120a can collect IL-2 and second particle 120b can collect VEGF, as shown in more detail in the schematic of step 802. Fluorescent detection of collected IL-2 and VEGF can be used for isolating and sorting high and low IL-2 secreting cells and high and low VEGF secreting cells via FACS, depicted in the schematic of step 804. Process 800 can end at cell expansion of high and low IL-2 secretor cells and/or high and low VEGF secretor cells as shown in step 806 or can continue further for validation of the respective secretors via ELISA or any other suitable validation technique. In particular, using two different particles 120a, 120b for creating composition 100b four populations of cells: IL2⁺/VEGF⁺, IL2⁺/VEGF⁻, IL2⁻/VEGF⁺, and IL2⁻/VEGF⁻. Although not shown, additional particles can be used to generate more than 2 or 4 populations of cells. When using more than two particles, the cells can be isolated based on a multi-color experiment or on a serial isolation process. When multiple particles are used for different secreted molecule collection, a common targeting unit can also be used.

When using two different particles, the detecting unit can specifically bind to the secreted molecule. In some instances, there may be no secreted molecule from the cell and therefore no collection. In such a case, the detecting unit will not be attached to the particle or the composition and is indicative of no secretion from the cell. In some other instances, such as negative secretion, the particle can still remain on the cell for sorting, indicating the correct particle-cell binding or even the cell activation.

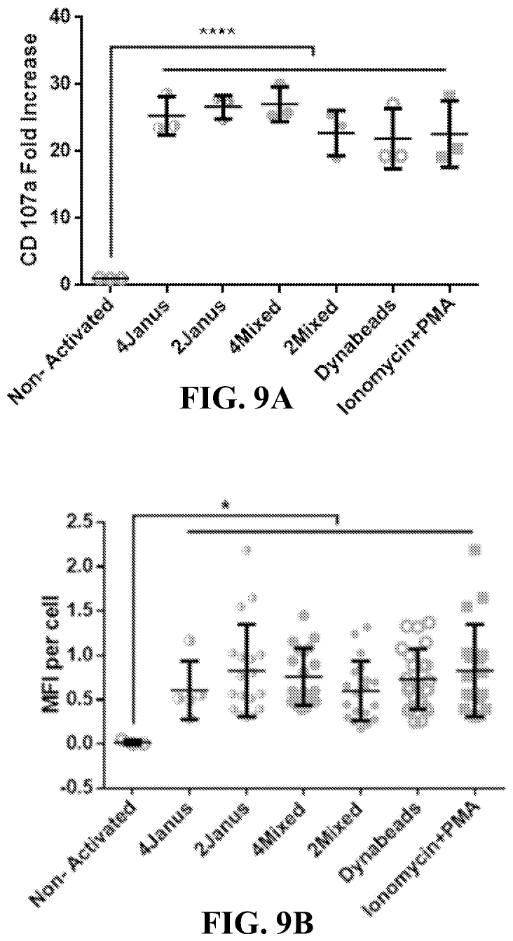

FIG. 9A provides a plot showing CD 107a fold increase for various particles 120. In particular, both particle types of 2 μm and 4 μm in diameter demonstrated similar activation of Jurkat cells, assessed by CD107a expression compared with common stimulatory protocols such as Ionomycin+PMA and Dynabeads. The mean fluorescence intensity (MFI) was determined for single cells using the ZEN 2.3 SP1 (Zeiss) software, where the particle area was excluded and the cell surface intensity was analyzed, as shown in FIG. 9B.

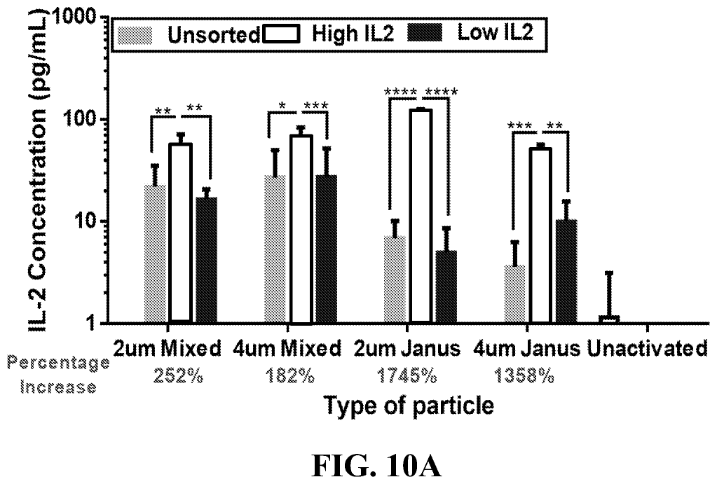

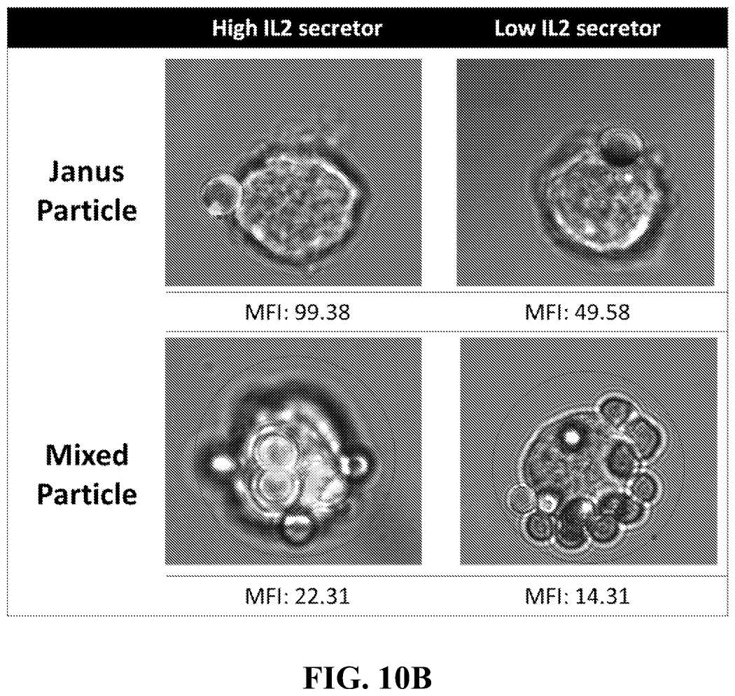

FIG. 10A provides a plot of IL-2 concentration for various particles and percentage increase of IL-2 concentration between unsorted cells and isolated high and low IL-2 secreting cells, where *, *, ***, and **** represents p=0.0130, p<0.01, p<0.001, and p<0.0001, respectively.

FIG. 10B provides confocal images and MFI of the detection antibody of an isolated high secreting cells and a low secreting cells.

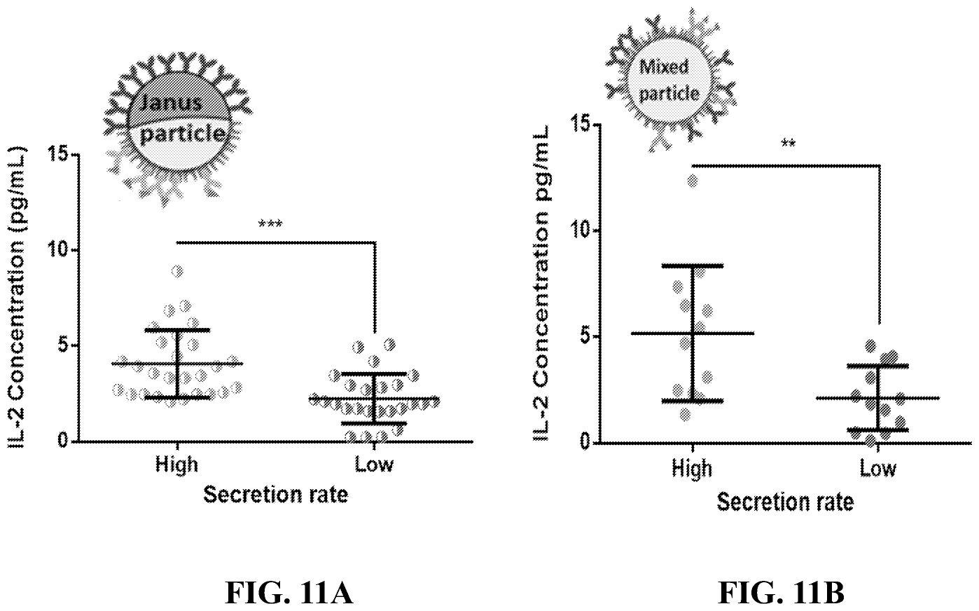

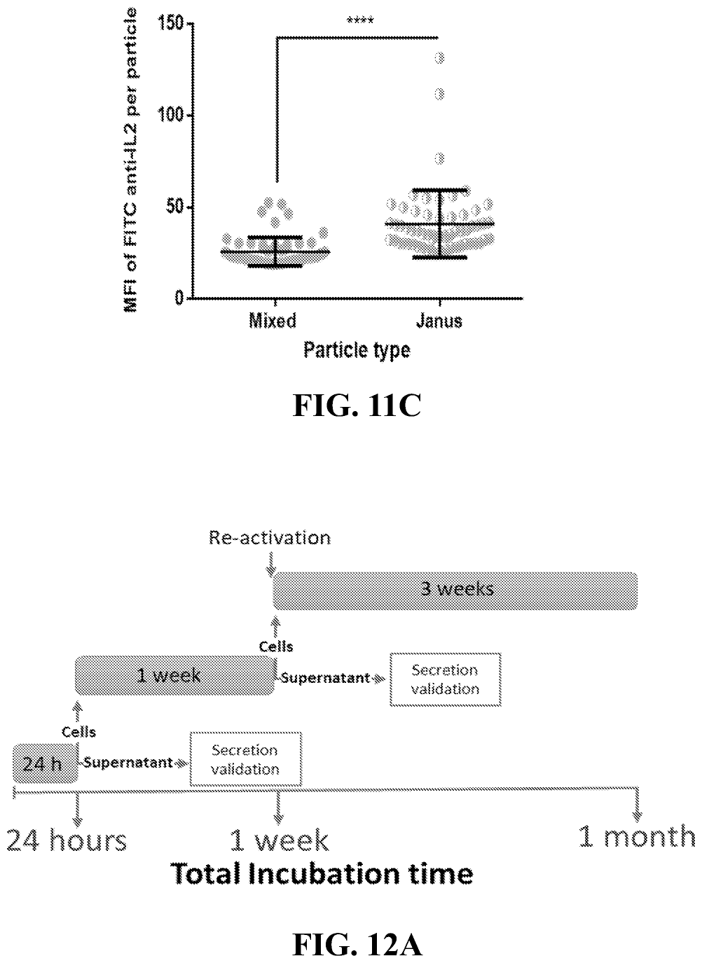

FIGS. 11A and 11B provide analysis of single FACS-sorted cells and IL-2 concentration for high and low secretors for various particle coating types including Janus particles (FIG. 11A), mixed particles (FIG. 11B). FIG. 11C provides detection antibody intensity on Janus and mixed particles for single cells, where *, ***, and **** represents p<0.01, p<0.001, and p<0.0001, respectively.

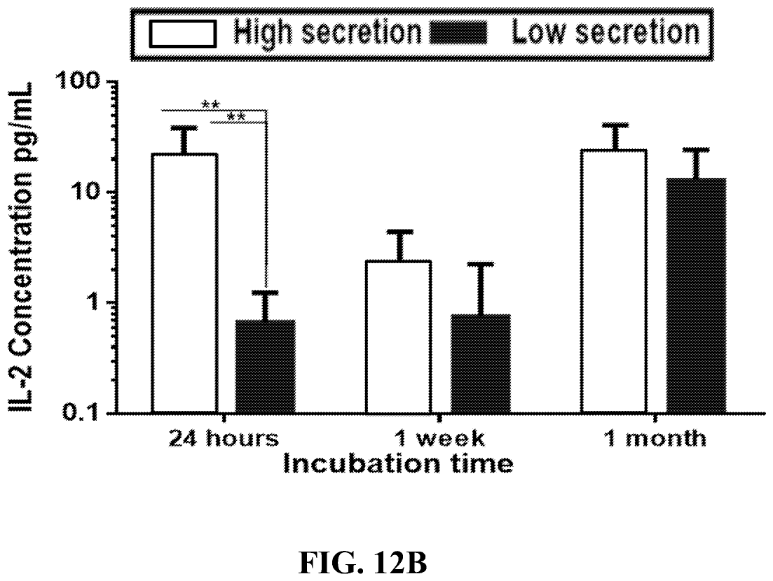

FIGS. 12A and 12B provide inheritability of IL-2 secretion in Jurkat cells. In particular, FIG. 12A is a timeline of an example experiment for evaluating IL-2 concentration of 1,000 cells isolated as high and low IL-2 secreting cells and unsorted cells incubated for 24 hours, one week, and one month after isolation using Janus particles. FIG. 12B provides IL-2 concentration for 1,000 cells isolated as high and low IL-2 secreting cells and unsorted cells incubated for 24 hours, one week, and one month after isolation using Janus particles, where ** is equal to p<0.0001. IL-2 concentration was not evaluated for cells isolated via Janus particles incubated for one week.

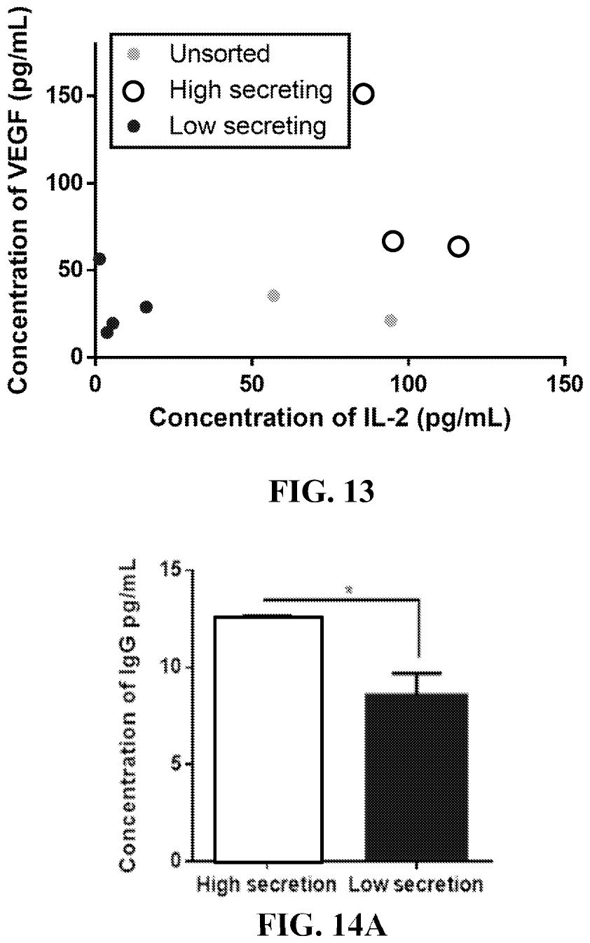

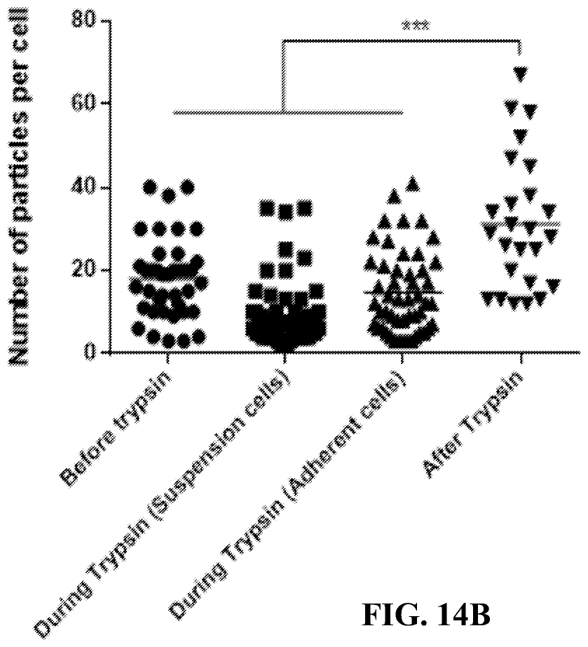

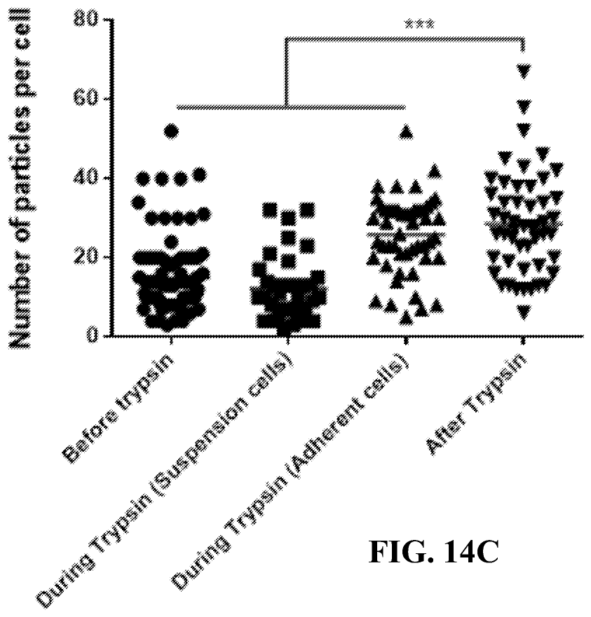

FIG. 13 provides a plot of concentration of IL-2 versus concentration of VEGF of unsorted, high secretors, and low secretors isolated by the particles. FIG. 14 provides a plot of concentration of IgG from high secretors and low secretors isolated by the particles.

As shown in FIGS. 14A and 14B, a significantly higher number of particles per cell can be found after trypsinization than when particles were added before or during cell trypsinization. In some examples, cells can be trypsinized first and allowed to adhere before adding particles. Any unbound particles can be removed by washing. Cells can then be incubated during 4 days for VEGF secretion and collection. High and low VEGF secreting cells can be sorted via FACS and validated the secretion using ELISA specific for VEGF.

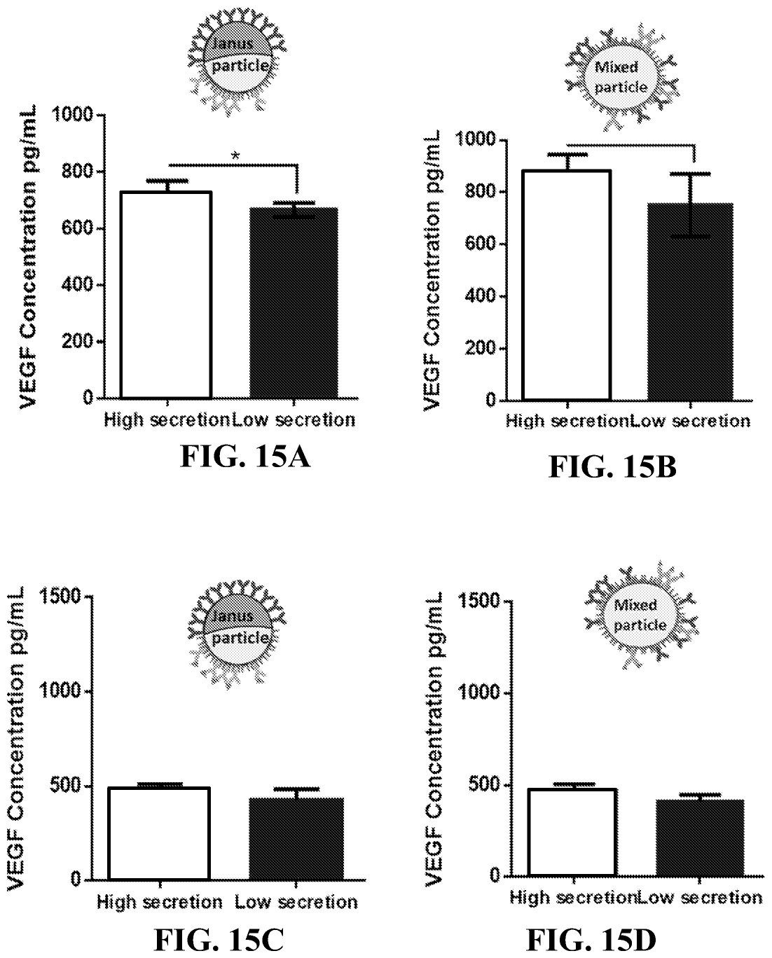

FIGS. 15A through 15D provide plots of concentration of VEGF from high secretors and low secretors isolated by various particles including Janus particles after 4 days of incubation (FIG. 15A), mixed particles after 4 days of incubation (FIG. 15B), and Janus particles after 8 days of incubation (FIG. 15C), and mixed particles after 8 days of incubation (FIG. 15D).

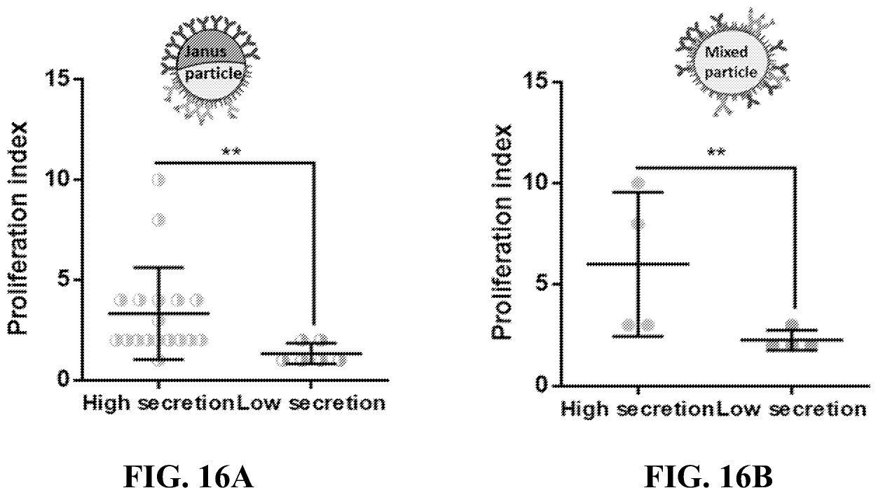

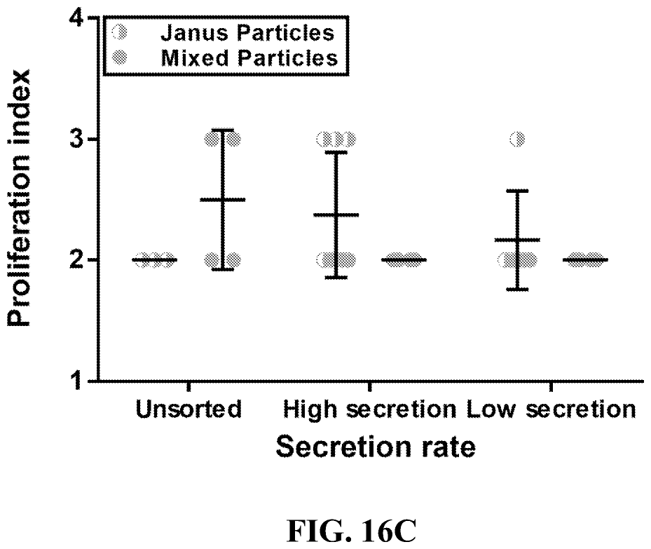

FIGS. 16A and 16B provide plots of proliferation index of isolated high and low VEGF secreting cells isolated with Janus particles (FIG. 16A) and mixed particles (FIG. 16B) after 4 days of incubation. In particular, the functional assay of isolated high VEGF secreting cells shows higher proliferation faster.

FIG. 16C provides a plot of proliferation index of cells isolated with Janus particles and mixed particles after 8 days of incubation.

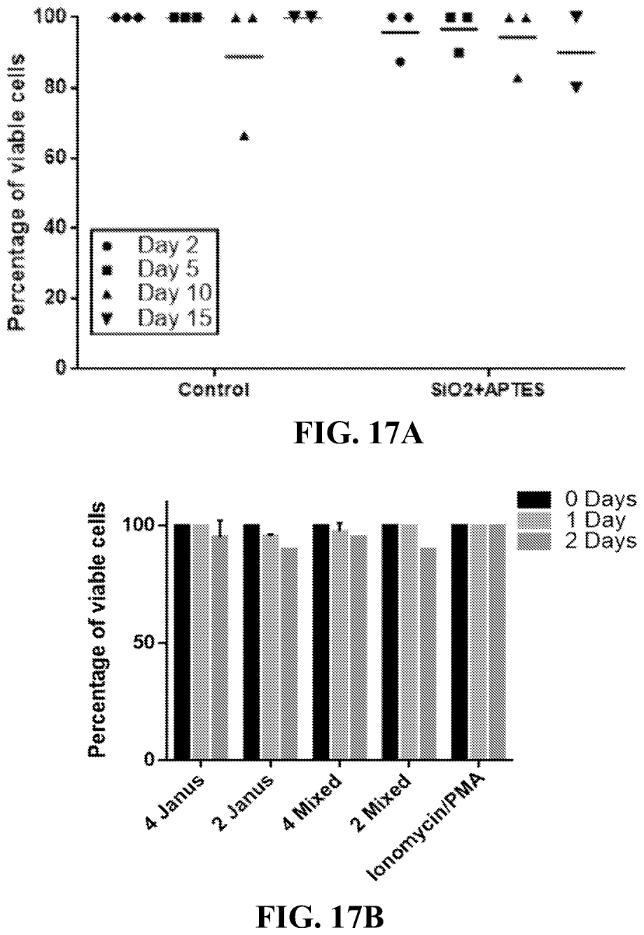

FIGS. 17A and 17B provide plots of percentage of viable cells indicating no toxic effect of particles. As shown, experiments evaluated the viability response of TIB 147 cells with 4 μm APTES-functionalized particles conjugated with anti-CD44 at different incubation times (n=3).

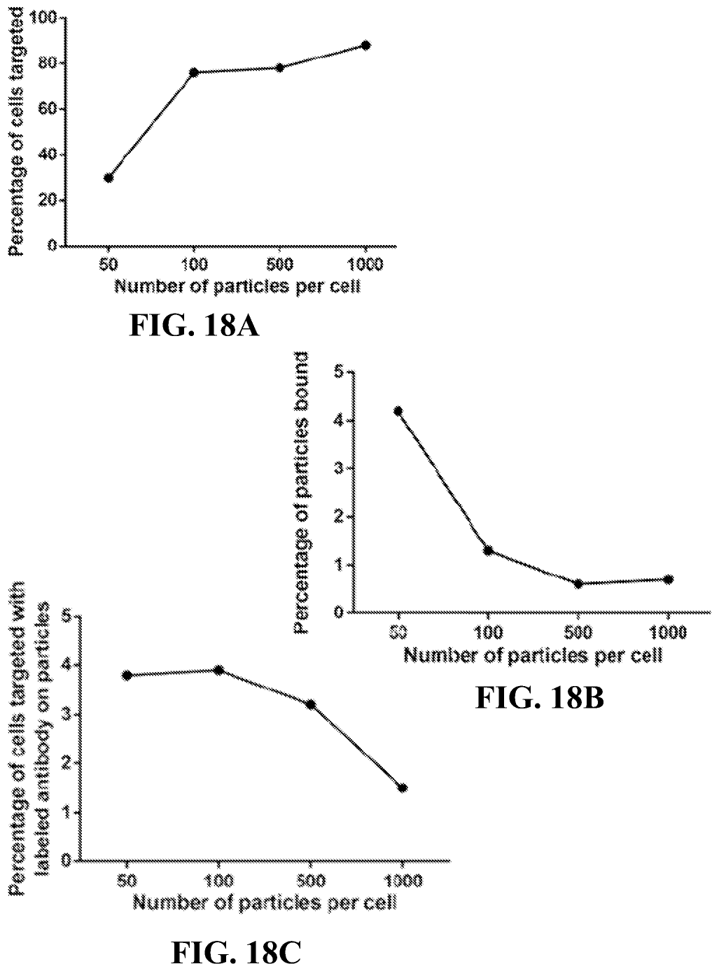

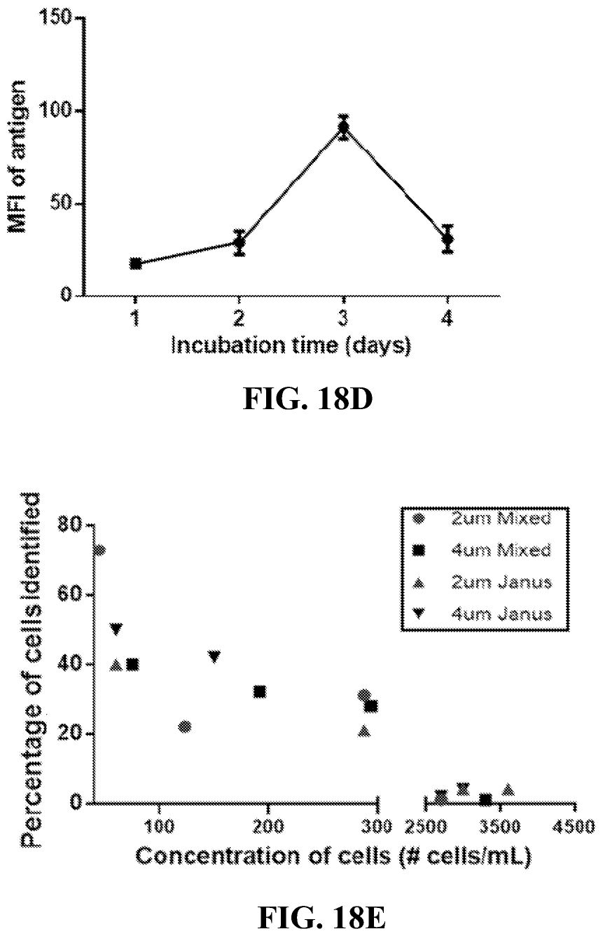

FIGS. 18A through 18E provide plots demonstrating optimal conditions for efficient cells targeting with labeled antibody maximizing antigen intensity per particle. In an example embodiment, experiments evaluated the response of 4 μm Janus particles with TIB147 cells. As shown in FIG. 18A, different ratios of particles per cell can be evaluated to determine efficiency of cells targeting. FIG. 18B shows the percentage of particles bound to cells for number of particles per cell. FIG. 18C provides percentage of cells targeted with labeled antibody on particles for number of particles per cell. (D) Antigen MFI of labeled antibodies on particles bound to cells (E) Efficiency of positive cells identified modifying the concentration of cells per mL using 2 μm and 4 μm using heterofunctional particles. Positive cells are cells correctly identified with the specific antigen in a mixture of cells.

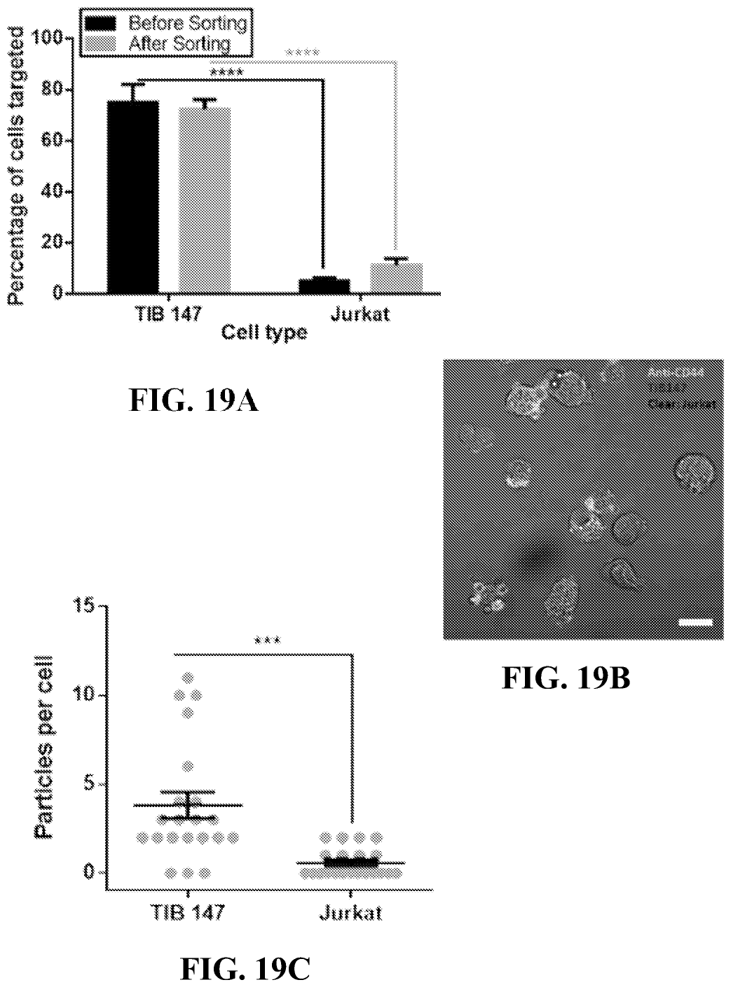

FIGS. 19A and 19C provide plots of percentage of cells targeted before and after sorting of particles based on cell type, specifically for either blue cell tracker, TIB 147, and Jurkat cells, and particles per cell after sorting the particle for either TIB 147 or Jurkat cells. FIG. 19B is a confocal image of Jurkat and blue cell tracker, TIB147, targeted by a 4 m mixed particle with FIC Anti-CD44.

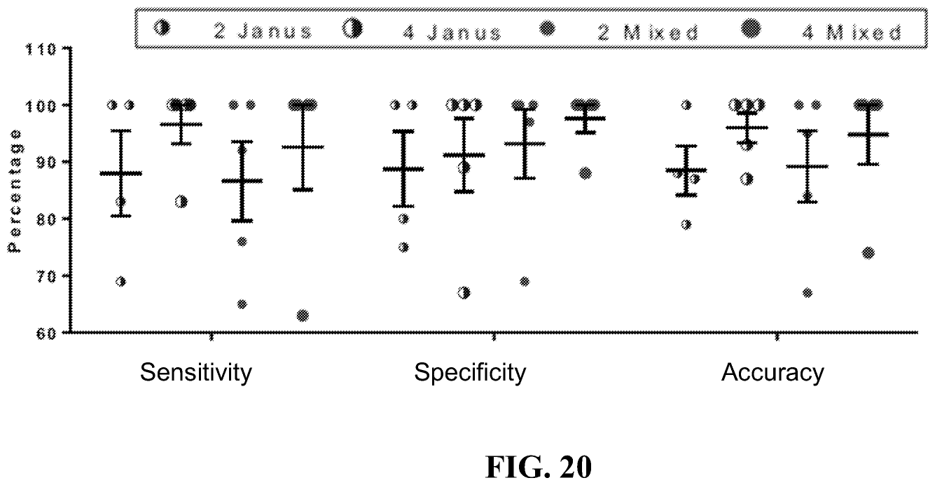

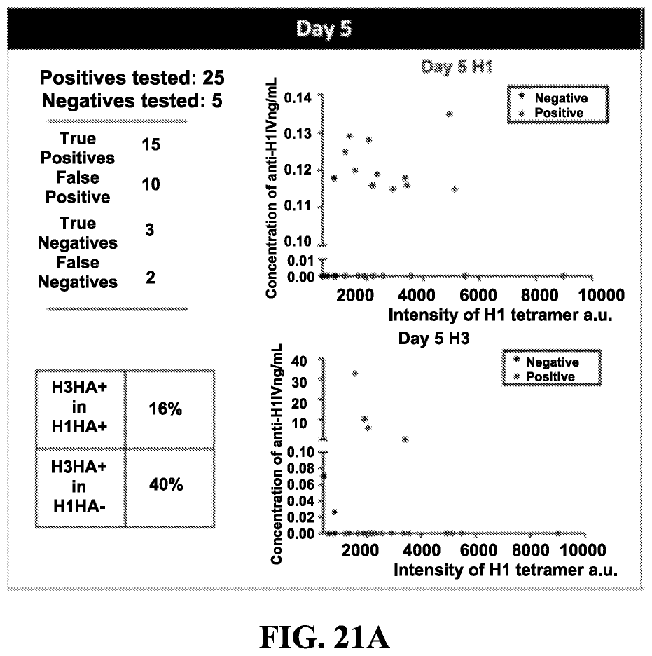

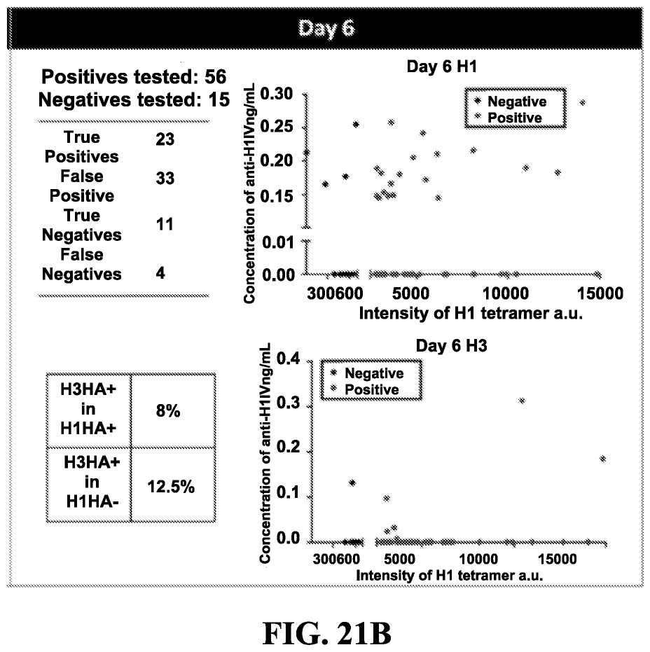

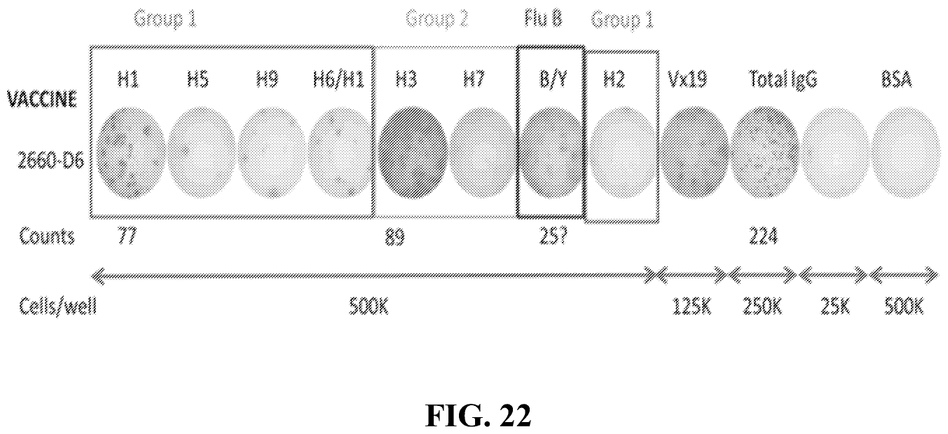

FIG. 20 provides a plot of percentage of sensitivity, specificity, and accuracy for either Janus particles or mixed particles of various sizes. FIGS. 21A and 21B provide correlation of intensity of H1 and H3 tetramer and concentration of H1IV and H3IV antibody using particles. FIG. 22 provides an example ELISPOT result for a sample patient “2600” for day 6 post vaccination.

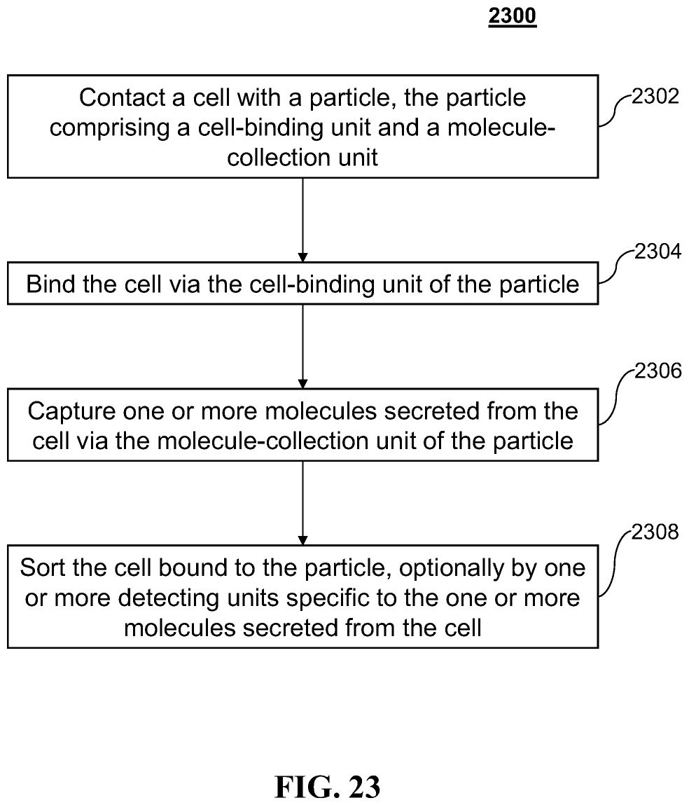

FIG. 23 is a flow diagram outlining steps outlining a method 2300 of isolating cells with a single particle. In some embodiments, method 2300 can include contacting 2302 the cell with a particle. The particle can include a cell-binding unit and a molecule-collection unit. Method 2300 can further include binding 2304 the cell via the cell-binding unit of the particle. Method 2300 can further include capturing 2306 one or more molecules secreted from the cell via the molecule-collection unit of the particle. In addition, method 2300 can include sorting 2308 the cell bound to the particle, optionally by one or more detecting units specific to the one or more molecules secreted from the cell. Method 2300 can end after step 2308 or can continue further to include releasing the cell from the particle, expanding the cell, detecting a quantity of the one or more molecules secreted from the cell, optionally using the one or more detecting units specific to the one or more molecules secreted from the cell, and/or separating a low-secretion cell from a high-secretion cell based on the quantity of the one or more molecules secreted from the respective cell.

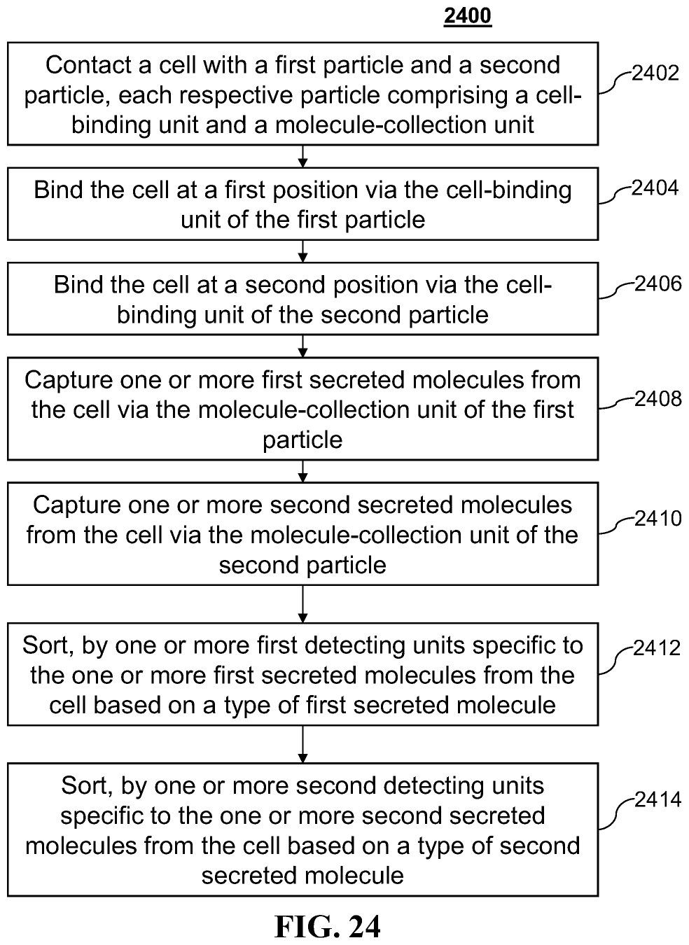

FIG. 24 is a flow diagram outlining steps outlining a method 2400 of isolating cells with more than one particle. In some embodiments, method 2400 can include contacting 2402 the cell with a first particle and a second particle, each respective particle comprising a cell-binding unit and a molecule-collection unit. The cell-binding unit and the molecule-collection unit on the respective particle can include at least one of a molecule, an antibody, or a protein. The cell-binding unit and the molecule-collection unit on the first particle can be different than the cell-binding unit and the molecule-collection unit on the second particle. Method 2400 can also include binding 2404 the cell at a first position via the cell-binding unit of the first particle. Method 2400 can further include binding 2406 the cell at a second position via the cell-binding unit of the second particle. Additionally, method 2400 can include capturing 2408 one or more first secreted molecules from the cell via the molecule-collection unit of the first particle and capturing 2410 one or more second secreted molecules from the cell via the molecule-collection unit of the second particle. Method 2400 can further include sorting 2412, by one or more first detecting units specific to the one or more first secreted molecules from the cell based on a type of first secreted molecule. Finally, method 2400 can include sorting 2414, by one or more second detecting units specific to the one or more second secreted molecules from the cell based on a type of second secreted molecule. Sorting 2412, 2414 the cell can be based on a quantity of the first secreted molecule and second secreted molecule from the cell using the one or more first detecting units and one or more second detecting units.

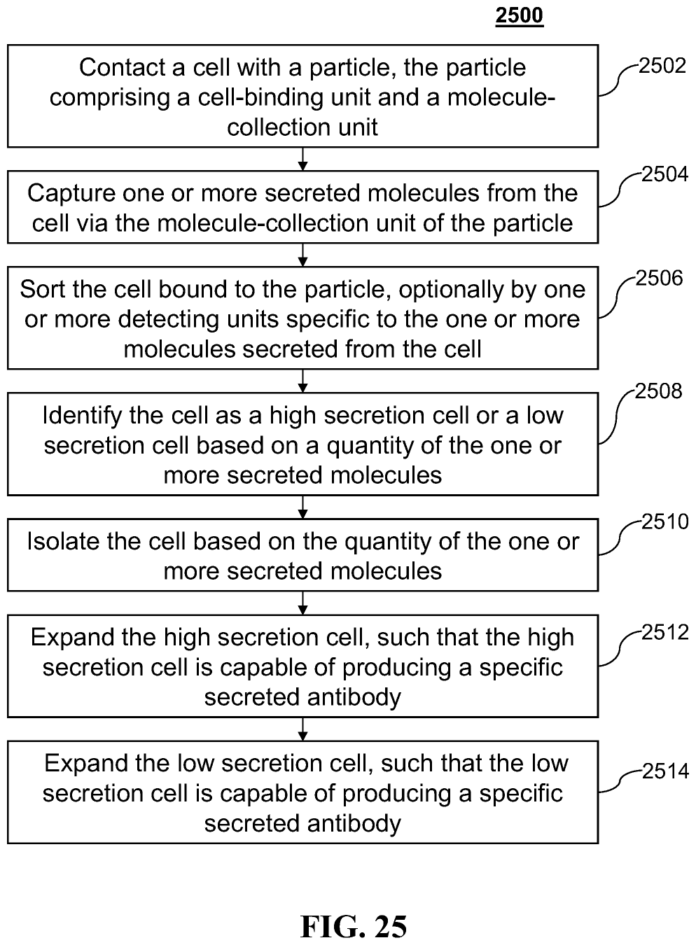

FIG. 25 is a flow diagram of outlining a method 2500 of producing secreted molecules. Method 2500 can include contacting 2502 a cell with a particle, the particle comprising a cell-binding unit and a molecule-collection unit. Method 2500 can also include capturing 2504 one or more secreted molecules from the cell via the molecule-collection unit of the particle, sorting 2506 the cell bound to the particle, optionally by one or more detecting units specific to the one or more molecules secreted from the cell. Method 2500 can end at step 2506 or can further include identifying 2508 the cell based on a type of the one or more secreted molecules, isolating 2510 the cell based on the type of the one or more secreted molecules, and/or expanding 2512 the cell based on the type of the one or more secreted molecules, such that the cell is capable of producing a specific secreted antibody or other molecule. In some embodiments, identifying 2508 the cell as a high secretion cell or a low secretion cell can be based on a quantity of the one or more secreted molecules. In some examples, isolating 2510 the cell can be based on the quantity of the one or more secreted molecules. Further, method 2500 can further include expanding 2512 the high secretion cell, such that the high secretion cell is capable of producing a specific secreted antibody. Alternatively, or in addition thereto, method 2500 can include expanding 2514 the low secretion cell, such that the low secretion cell is capable of producing a specific secreted antibody.

In some embodiments, the methods described herein can include a screening process where cells can be positioned in a well plate, evaluated for secretion, and then selected or isolated based on the amount of and/or type of secretion produced by the respective cell population. As described supra, the method can provide a simplified and high throughput screening process such that hundreds of cells can be evaluated and isolated for secreted molecules.

The following examples further illustrate aspects of the present disclosure. However, they are in no way a limitation of the teachings or disclosure of the present disclosure as set forth herein.

### EXAMPLES

### Example 1. Materials

Silica microspheres with diameters of 1.0 μm, 2.0 μm, and 4.0 μm, as well as carboxylated polystyrene microspheres with a diameter of 7 μm, were purchased from Bangs Laboratories (Fishers, IN). Coupling reagents, including (3-Aminopropyl)triethoxysilane (APTES), 1-Ethyl-3-(3-dimethylaminopropyl)carbodiimide (EDAC), acetone, phosphate buffered saline (PBS), PolyLink Wash/Storage Buffer, PolyLink Coupling Buffer, Dulbecco's Modified Eagle's Medium (DMEM), Iscove's Modified Dulbecco's Medium (IMDM), gentamicin, penicillin/streptomycin, L-glutamine 200 mM, 10% sodium pyruvate, concanavalin A Alexa Fluor 488 were purchased from Sigma-Aldrich (St. Louis, MO) and fetal bovine serum (FBS) from Atlanta Biologicals (Atlanta, GA). Bovine serum albumin (BSA) were purchased from Sigma-Aldrich (St. Louis, MO) for use to passivate bead surfaces. Thiol-poly(ethylene glycol)-biotin, a heterobifunctional PEG derivative, was purchased from Nanocs (New York, NY). Human Bcl6 peptide was purchase from Abcam (Cambridge, UK). Anti-mouse/human Anti-CD44 antibody conjugated with APC fluorophore was purchased from Biolegend (San Diego, CA). Lightning-Link® Streptavidin was obtained from Expedeon (San Diego, CA). Protein G was purchased from Protein Specialists (East Brunswick, NJ). TIB 147 and BCL6 hybridomas were obtained from ATCC and Iowa University, respectively. PE-labeled H1HA tetramer as gift from Lund lab at Emory University (Atlanta, GA).

### Example 2. Cell Culture of Hybridomas and Antibody Secreting Cells

TIB 147 and BCL6 hybridoma cells were chosen as each produces a distinct type of antibody useful for evaluating the specificity of collection and detection of antibodies. TIB 147 hybridoma cells produce anti-concanavalin A and BCL6 hybridoma cells produce anti-BCL6, respectively. TIB147 cells were purchased from ATCC (Manassas, VA) and BCL6 hybridomas were purchased from the Iowa University Hybridoma Bank (Iowa City, IA). TIB 147 cells were cultured in complete media composed of DMEM supplemented with 10% fetal bovine serum and 0.1% penicillin/streptomycin. BCL6 cells were cultured in IMDM, 20% fetal bovine serum, 2 mM of L-glutamine, 1 mM of sodium pyruvate, 50 μg/mL of gentamicin, and 0.1% penicillin/streptomycin. Antibody secreting cells were cultured in complete media composed of RPMI supplemented with 10% Fetal Bovine Serum, and 0.1% penicillin/streptomycin. Cells were grown in a humidified incubator at 37° C. supplemented with 5% CO₂. ASCs were single-cell incubated in 96 well-plates over 6 days for further analysis. Hybridoma cells were expanded in cell culture T-75 flasks over three days to a final concentration of 1×10⁶/mL.

### Example 3. Preparation of Janus and Mixed Heterofunctional Particles

Janus particles were fabricated using a modification of the protocol proposed by Tang et al. See Tang, J. L., Schoenwald, K., Potter, D., White, D., & Sulchek, T. (2012). Bifunctional Janus Microparticles with Spatially Segregated Proteins. Langmuir, 28(26), 10033-10039. Particles were washed repeatedly with deionized water and suspended in 100% ethanol in a ratio of 1:12 (particle stock solution: ethanol) for 1 μm particles, 1:10 for 2 μm, 1:8 for 4 μm and 1:5 for 7 μm. Droplets of 8 μL of suspension were spotted onto glass slides and dried at room temperature by shaking the slides on an orbital rotator set at 200 rpm. Particles were then coated with a layer of titanium adhesion layer followed by gold a using a metal evaporation process (CHA E-Beam Evaporator). The titanium and gold were deposited respectively to a final thickness of 50 Å and 100 Å for 1 μm particles, 100 Å and 500 Å for 2 μm, 500 Å and 1000 Å for 4 μm, and 1000 Å and 1,500 Å for 7 μm with a rate of 1 Å/s. After the gold deposition, the glass slides were placed into 50 mL centrifuge tubes filled with deionized water and gently sonicated (Haier Ultrasonic Cleaner) for 5 minutes to remove the gold-coated particles from the glass substrate.

Antibody labeled with streptavidin was conjugated to the gold hemisphere that was modified via thiol-PEG-biotin. Protein G molecules were adhered to the substrate hemisphere as shown in FIG. 2B. 1×10⁶ Janus particles were incubated for 4 hours with 1 mM solution of thiol-PEG-biotin (500 μL) in PBS at room temperature in a spinner. Silica Janus particles were washed with acetone and functionalized with 2% APTES solution in acetone; whereas polystyrene Janus particles were washed with PolyLink Coupling Buffer and functionalized with 0.2 mg/mL of EDAC and PolyLink Coupling Buffer. APTES or EDAC excess was removed through PBS washes and protein G was added for 1 hour at room temperature. Particles were then washed with PBS, resuspended with 1% BSA Blocking Buffer for 1 hour, and washed with PBS twice to remove excess of BSA. After functionalization of the silica/polystyrene hemisphere, particles were incubated for 1 hour at room temperature with anti-CD44 (5 μg) conjugated to streptavidin using Lightning-Link Streptavidin Conjugation Kit following the manufacturer's instructions. APC Anti-mouse/human CD44 is the targeting antibody specific for TIB 147 and BCL6 hybridomas. Functionalized 4 μm silica Janus particles were also coated with APC streptavidin and FITC antibody following Protein G adsorption to validate the functionality of the two surfaces. Flow cytometry (Accuri C6) was used to verify the intensity distribution of the fluorophore conjugated proteins in a sample containing 1×10⁵ particles. The mean fluorescent intensity (MFI) was determined for the sample conjugated with the two proteins and three controls: Janus particles without protein conjugation, particles coated with APC streptavidin on the gold hemisphere, and particles conjugated with FITC antibody on the silica hemisphere. A gating strategy was performed to exclude debris and doublets, and the MFI of the samples was evaluated in the FL-1 and FL-4 channels, corresponding to the FITC and APC, respectively. To demonstrate the spatial segregation of the two proteins onto the Janus Particle, images were acquired using confocal laser microscopy (Zeiss LSM 510 VIS Confocal Microscope). The methods to determine the optimal concentration of antibody on mixed particles, number of particles per cell, particle incubation time, and particle toxicity are described in the Supplementary Information.

### Example 4. Determining the Optimal Concentration of Cells to Minimize Molecular Cross-Talk of Collected Antibody

Cell concentration that optimized the percentage of correctly identified cells by the specific antibody in a mixture of cells was determined, which reduced molecular cross-talk. BCL-6 and TIB 147 hybridoma cells were combined in equal number and attached to heterofunctional particles consisting of Anti CD44 to target both cells and protein G to collect secreted antibody. Cells successfully bound to particles were sorted via FACS and incubated for 3 days at various concentrations in a glass bottom dish. FITC-concanavalin A (5 μg) and APC-BCL6 (5 μg) were added to evaluate the specificity of collected antibody by particles bound to TIB 147 and BCL-6 cells. The percentage of cells correctly identified by their secreted antibody was completed by dividing the number of BCL-6 cells with APC BCL6 antigen or the number of TIB147 with FITC concanavalin A on the particles attached to the cell over the total number of each cell type in the sample. All cells were identified by counting using fluorescent confocal microscopy.

### Example 5. Determining the Optimal Particle Size to Avoid Detachment from Cells when Using FACS

Particle sizes ranging from 1.0, 2.0, 4.0, to 7.0 μm in diameter were evaluated, as well as compared Janus and mixed particles, to investigate if particles remain attached during FACS. Particles functionalized with protein G and APC anti-CD44 were combined with TIB147 cells in a ratio of 1:100 particles. Cells targeted by particles were sorted using FACS, incubated for 3 days in a bottom glass dish, and evaluated for antibody collection by adding FITC concanavalin A. The number of particles per cell before and after sort were calculated and the percentage of cells targeted and cells identified. The optimal particle size and particle configuration that improved cells identification and decreased the number of detached particles was determined. The number of particles per cell and the number of cells targeted and cells identified was determined using confocal microscopy and manual counting. Multiple comparison ANOVA was used to determine statistical significance of the variables. The methods to determine the optimal particle to cell ratio to avoid detachment of particles when using FACS are described in Example 18 below.

### Example 6. Comparison Between Janus and Mixed Particles to Target and Identify Cells

Mixed and Janus particles of 4 μm in diameter functionalized with protein G collector molecule and APC anti-CD44 targeting molecule were combined with TIB147 cells and in a ratio of 1:100 to evaluate the accuracy of particle binding. Targeted cells were sorted using FACS, and incubated cells for a period of 3 days. The collected antibody was stained by added FITC concanavalin A (5 μg). The percentage of particles bound to cells, the percentage of particles with anti-CD44, and the percentage of bound particles bound to cells with labeled antibody for both particles were determined. The percentage of cells targeted and cells identified with labeled antibody was also found. Janus and mixed particles were compared to target cells and collect antibody by confocal microscopy and manual counting. Using multiple comparison ANOVA, statistical significance of the variables was determined. The methods to compare the mean fluorescent intensity of labeled antibody between Janus and mixed particles were determined by combining 4 μm mixed and Janus particles functionalized with protein G and APC anti-CD44 with TIB147 cells in a ratio of 1:100 and sorted cells targeted using FACS. The cells were incubated for 3 days and FITC concanavalin A (5 μg) was added to measure the fluorescent intensity of labeled antibody. The mean fluorescent intensity of FITC concanavalin A was determined for the 50 bright particles bound to cells to compare the maximum collection of antibodies in Janus and mixed particles and evaluate the incorporation of two individual hemispheres present in the Janus. The MFI of each particle was calculated using the Zen Lite software. A t-test was used to determine the statistical significance of the antigen MFI between the two particles.

### Example 7. Evaluating Specific Targeting of Anti CD44 Particle to TIB147 (CD44+) and Jurkat (CD44−) Cells

The specificity of targeting was evaluated by adding particles with Anti-CD44, as the targeting molecule, in an equal mixture of CD44+ TIB147 and CD44− Jurkat cells. Cells and particles were mixed for 1 hour, sorted using FACS for targeted cells, and incubated in a glass bottom dish for confocal analysis. The number of particles per cell and the percentage of cells targeted before and after FACS implementation was calculated for both cell types by counting the events using confocal microscopy. Multiple comparison ANOVA and a t test was used to find the significance of the percentage of cells targeted and the number of particles per cell, respectively.

### Example 8. Evaluating the Specificity, Sensitivity, and Accuracy of Janus and Mixed Particles as Single Cell Antibody Collectors and Detectors

The selectivity, sensitivity, and the accuracy of the bifunctional particles was evaluated as single cell secreted antibody collectors and detectors. Anti-BCL6 and anti-concanavalin A were detected in a sample containing both TIB147 and BCL6 hybridoma cells. 2 μm and 4 μm Janus and mixed particles functionalized with protein G were and unstained anti-CD44 were combined with a mixture of TIB 147 and BCL6 cells in equal amounts and in a ratio of 1:100 cell to particle ratio. Targeted cells were sorted using FACS and incubated for 3 days in a glass bottom dish of 25 mm with cell media (300 μl) in a humidified incubator. FITC concanavalin A (5 μg) and APC BCL6 (5 μg) were added to calculate the percentage of particles bound to TIB 147 and BCL-6 that collected concanavalin A and BCL6 antibody. The cells with labeled antibody were identified using confocal microscopy and manual counting and determined the percentage of positive and negative cells over the total number of cells targeted. Positive cells were defined as cells whose particles attached collected only the local antibody. Conversely, cells whose particles did not collected the local or the antibody secreted by the other cell line were denoted as negative cells. Multiple comparison ANOVA was used to determine statistical significance of positive and negative cells and to determine the statistical difference between the two particle configurations in each condition and performed multiple t tests to find the significance of particle size in each configuration.

The selectivity, sensitivity, and accuracy was determined using a contingency table, shown in FIG. 20. TIB 147 was evaluated as the positive test and BCL6 as the negative test. Therefore, true positives events refer to particles attached to TIB 147 that collected only anti-concanavalin A, whereas true negatives to particles attached to BCL6 that collected only anti BCL-6. Similarly, false positives refer to particles attached to TIB 147 that collected anti-BCL6 or both antibodies, while false negatives refer to particles attached to BCL6 that collected anti-concanavalin A or both antibodies. The average of the percentage of positive and negative cells was used to calculate the parameters in the contingency table. FIG. 3B shows the contingency table parameters for the contingency table and equations used for sensitivity, specificity, and accuracy for an example experiment with TIB147 and BCL6 to identify anti-concanavalin A and anti BCL6.

### Example 9. Collection of B Cells from Vaccinated Patients

Peripheral blood was collected from a healthy 32-year-old female at 5- and 6-days post receiving 2019-2020 quadrivalent influenza vaccine (QIV), using BD Vacutainer® Sodium Heparin tubes. All research was approved by the Emory Institutional Review Board and performed in accordance with all relevant guidelines/regulations and informed consent was obtained from all participants (IRB00057983). The sample was diluted in an equal volume of PBS and layered over Leucosep™ tubes previously filled with Lymphocyte Separation Media. The tubes were centrifuged at 1000×g for 10 minutes and collected the peripheral blood mononuclear cells (PBMC) layer. The cells were resuspended in RPMI and centrifuged at 500×g for 10 minutes. The supernatant was removed and Gey's solution (155 mM NH₄Cl, 5 mL) was added for 3 minutes at 4° C. to lyse contaminating red blood cells. The Gey's solution was removed by washing the sample twice in RPMI. B cells were enriched using the EasySep™ Human Pan-B Cell Enrichment Kit (Stemcell Technologies) via negative selection following the manufacturer's instructions. Finally, the cells were counted by the TC20 cell counter (Biorad) using 0.4% Trypan Blue exclusion.

### Example 10. Isolation of Antibody Secreting Cells Specific to H1N1 Influenza Virus

ASC were isolated from enriched B cells by FACS using the following antibody panel against cell surface markers: IgD FITC, CD138 APC, CD3 BV711, CD14 BV711, CD19 PE-Cy7, CD38 V450, (BD Biosciences, Biolegend, Miltenyi Biotec). B cells were stained with the antibody cocktail for 20 minutes at 4° C., followed by washes with HBSS with 1% BSA to remove unbound antibodies. The stained B cells were combined with 1 μm mixed polystyrene particles conjugated with protein G and anti-CD27 APC-eFlour780 (eBioscience), as the targeting antibody in a 1:100 cell per particle ratio. Multiple centrifugations were performed at 100×g for 5 minutes in a microcentrifuge tube until the sample was free of unbound particles. Targeted ASC were counted via trypan blue exclusion and optical microscope and incubated overnight with RPMI with 10% FBS in a 96-well plate maintaining a concentration of 20,000 cells/well. Subsequently, Neuraminidase (5 U/mL) and human IgG (5 μg/μL; Jackson Immunoresearch) were added to the incubated cells for 30 minutes at 37° C., concurrently. Neuraminidase was added to cleave sialic acid groups on the surface of ASC to reduce unspecific binding of the H1-Hemagglutinin (H1HA) tetramer to the cells. IgG was added to block any further protein G interaction binding. All wells were combined and added PE-labeled H1HA tetramer at a dilution of 1:100 per volume in staining buffer. The cells were sorted for H1HA-specific IgG and ASC following the gating strategy for isolation of H1IV ASCs, where in a first sort of cells, a second sort of CD19⁺, CD3⁻, and CD14⁻, a third sort of Particles⁺CD38⁺, a fourth sort of H1IV⁻, and a fifth sort of H1IV⁺. Cells were collected on Aria II (BD Biosciences) configured to detect 6 fluorochromes. Analysis was performed using FlowJo software (Treestar, Inc. version 8.7.1).

### Example 11. Validation of Collected H1 Influenza Virus Antibody Secreting Cells

To validate H1HA-specificity, H1HA-specific ASCs were single cell sorted and cultured for 6 days in plasma cell survival medium (PCSM) with 200 ng/mL APRIL (R&D Systems). After culture, the supernatant of each well was divided for ELISA measuring H1HA-specific IgG and H3HA-specific IgG. High-binding 96 well plates were coated with 1 μg/mL of H1-HA (A/California/04/2009 (H1N1)pdm09; BEI Resources NR-15749) and H3-HA (A/New York/55/2004 (H3N2); BEI Resources NR-19241) by adding 100 μL to each well for 2 hours at room temperature on a shaker. For total IgG, 100 μL of Goat anti-human IgG (Jackson Immunoresearch) was added to plates at 2 μg/mL. 200 μl/well of SuperBlock™ Blocking Buffer was added for 1 hour on a shaker. After incubation, plates were washed with PBS with 0.1% Tween (PBST) 5 times. Samples and standards (CR9114 for HA-specific ELISA) were added for 1 hour on a shaker. Plates were washed 5 times and incubated for 1 hour with anti-Human IgG (Fc-specific; Sigma) detection antibody at a concentration of 50 ng/ml. After 5 washes, the alkaline phosphate substrate was added, and ELISA plates were read using the Biotek Synergy H4 micro plate reader.

### Example 12. Correlation of the PE-Labeled H1HA Tetramer Intensity of Single Cell-Sorted ASC and the Concentration Detected Via Single Cell ELISA

The antigen intensity of single cell-sorted ASC was compared and analyzed for the concentration of H1HA-specific and H3HA-specific IgG via single cell ELISA. The PE intensity of each cell sorted was evaluated using the index sort function of Aria II (FACS DIVA version III).

### Example 13. Evaluate the Specificity, Sensitivity, and Accuracy of Mixed Particles as H1IV ASC Detectors

The selectivity, sensitivity, and the accuracy of the particles as detectors of H1HA-specific antibody secreting cells was evaluated. A positive test is defined as an H1HA tetramer positive cell by flow cytometry and the negative test is defined as an H1HA tetramer negative cell. True positive and false positive samples refer to H1HA tetramer⁺ cells with positive and negative signal from H1HA via ELISA, respectively. False positives could be unspecific binding of antigen or a cell that did not produce antibody (e.g. died). Similarly, true negative and false negative samples refer to H1HA tetramer cells with negative and positive signal from H1HA via ELISA, respectively. The percentage of cells that made anti-H3HA antibodies by ELISA that were sorted as H1HA⁺ and HiHA⁻ was determined.

### Example 14. Influenza-Specific Antibody Secreting Cell (ASC) ELISpot

Several 96-well ELISpot plates (Millipore, MSIPN4W50) were coated at 37° C. for 2 hours with 1 μg/mL of H1-HA (A/California/04/2009 (H1N1)pdm09; BEI Resources NR-15749), H3-HA (A/Brisbane/10/2007 (H3N2); BEI Resources NR-19238) or 10 μg/mL of Goat anti-human IgG (H+L) (Life Technologies). 2% Bovine Serum Albumin (BSA, MP Biomedicals) in sterile PBS was used as an irrelevant antigen for control. The plates were blocked with RPMI with 10% FBS for 2 hours and incubated at 37° C. for 18-20 hours with 500,000 PBMC for the HA antigens and BSA. For total IgG we added 250,000 and 25,000 PBMC per well. After incubation, cells were aspirated, and plates were washed with PBST 6 times. Antigen specific antibodies bound to the plate were detected with alkaline phosphatase-conjugated anti-human IgG antibody (Jackson Immunoreseach) for 2 hours and developed with VECTOR Blue, Alkaline Phosphatase Substrate Kit III (Vector Laboratories). The spots per well were counted using the CTL immunospot reader (Cellular Technologies Ltd). For analysis, all samples had background spots from the BSA control wells subtracted (24).

### Example 15. Statistical Analysis

Statistical analysis was performed in Graphpad Prism (La Jolla, CA) using t-tests, one way, or two-way ANOVA to determine significance of variables. Post-hoc Tukey-Kramer HSD testing was performed to determine significance. Data are represented using mean±SEM.

### Example 16. Validation of the Two Hemispheres in Janus Particles

The Janus particles were verified to maintain spatial segregation of the conjugated CD44 and Protein G by recording the MFI from fluorescent APC-streptavidin conjugated to the gold side and FITC antibody bound to protein G conjugated to the silica side, as well as controls. FIGS. 4A through 4D show flow cytometry data that demonstrates the correct functionalization of both hemispheres while the images acquired with confocal laser microscopy and bright field microscopy confirm the spatial segregation of the bifunctionalized surfaces. In particular, APC-streptavidin was conjugated on the gold hemisphere and FITC antibody bound to Protein G on the silica hemisphere. 1×10⁵ particles were evaluated with a scale of 4 μm.

### Example 17. Evaluate Cell Health when Bound to Functionalized Particles

The viability of TIB147 cells were evaluated after incubation of anti-CD44 particles. FIG. 17 shows no statistical difference of percentage of viable cells between the conditions of different incubation times and the control with no particles, demonstrating no cytotoxic effect of the functionalized and conjugated particles. The impact of functionalized particles in the viability of FACS sorted B cells was also found to undergo a decrease of 14% compared with the control group.

### Example 18. Determining Optimal Concentration of Targeting Antibody in Mixed Particles

The optimal concentration of the targeting antibody in Janus particles and mixed particles was determined. FIG. 5C shows the effect of anti-CD44 concentration to target TIB147 cells (CD44+). At the highest concentration of targeting antibody, the percentage (defined as the positive events over the total events present in the sample) of cells targeted and number of particles with anti-CD44 increased, while the percentage of cells identified, and particles bound with labeled antibody decreased. It was found that 2 μg/μL of targeting antibody was optimal to allow sufficient targeting antibody binding while avoiding steric hindrance of the secreted antibody collection by protein G.

Due to the chance that attached particles will dissociate from cells during FACS processing, we tested 1:100 and 1:500 particle to cell ratios to investigate the robustness of processing, which includes FACS and the staining by antigen with several pipetting steps. We combined 4 μm Janus particles functionalized with protein G and APC anti-CD44 with TIB147 cells at several ratios of particles to cells. We used FACS to sort targeted cells in a bottom glass dish to avoid collection of antibodies on unbound particles leading to molecular cross-talk over the 3 day incubation. We calculated the number of particles per cell and the percentage of cells targeted before and after FACS to identify which particle to cell ratio increased cell targeting. The antibody collection by particles was evaluated by adding FITC concanavalin A to determine the number of particles per cell and the number of cells targeted with labeled antibody using confocal microscopy and manual counting. Multiple comparison ANOVA was used to determine statistical significance of the variables.

### Example 19. Comparing Particle Type and Size to Minimize Detachment from Cells During FACS

To determine if particles remain attached during the sorting, different particle to cell ratios and particle sizes were evaluated. A ratio of 1:500 cell per particles is favorable to avoid cell detachment. Larger particles collect more antibody, yet moderate sized particles remain attached during flow processing. Moreover, the detachment factor decreases the efficiency of cells identified when smaller and larger particles are used.

### Example 20. Optimal Particle to Cell Ratio, Cell Concentration, and Incubation Time for Efficient Cells Targeting and Cells Identification

The efficiency of particles to target and identify cells based upon antibody collection was evaluated using a particle to cell ratio in a range between 100:1 and 1000:1. The percentage of cells targeted, and particles bound demonstrates a proper targeting using 100 to 500 particles per cell. The percentage of cells identified was higher using 100 particles than 500 particles per cell.

The incubation time and cell concentration was optimized to efficiently identify cells at a high antigen intensity per particle. An incubation time up to 72 hours increases the MFI of antigen indicating a higher antibody collection. However, after 3 days a decline in cell viability was observed, causing a reduction of antigen MFI. A decrease in the efficiency of positive cells identified can result when more than 2,500 cells/mL are incubated, likely due to an increased molecular cross-talk from attached particles collecting antibody secreted from nearby cells.

### Example 21. Comparison Between Janus and Mixed Particles to Target and Identify Cells

To validate the targeting, collection of secreted antibodies, and fluorescent antigen labeling, 4 μm heterofunctional particles were combined with TIB 147 in a ratio of 100:1 particles per cell with an incubation time of 72 hours. As is shown in FIGS. 5B and 5C, Janus particles demonstrated improved targeting of cells, while mixed particles demonstrated improved antibody collection and detection. In some examples, about 14.3%+7.2% more antibody was collected by mixed particles than Janus particles (p<0.05).

### Example 22. Specificity of Particle Binding Using Mixed Particles in a Sample Containing TIB147 (CD44+) and Jurkat (CD44−) Cells

The specificity of targeting of mixed and Janus particles was evaluated by adding particles with Anti-CD44, as the targeting molecule, in a mixture of both TIB147 (CD44+) and Jurkat (CD44−) cells. Results illustrated FIGS. 19A through 19C indicates that the binding is significantly specific to the TIB147 cells and that the number of particles per cell demonstrated in FIG. 19C is significantly lower in Jurkat cells than TIB147.

### Example 23. Selectivity, Sensitivity, and Accuracy of Janus and Mixed Particles as Single Cell Antibody Collectors and Detectors in a Complex Mixture of Cells

The selectivity, sensitivity, and accuracy of 2 and 4 μm Janus and mixed particles as single cell secretion sensors was evaluated by labeling with BCL6 and concanavalin A in a sample containing both TIB147 and BCL6 hybridoma cells co-incubated with particles. When only concanavalin A antibody is detected, both particle sizes identify a higher number of positive cells and a lower number of negative cells. When both antibodies are detected, 2 μm particles achieve higher number of positive cells identified. T test indicates no significant difference between the two sizes in each condition. Similarly, two-way ANOVA demonstrates no statistical difference between Janus and mixed particles when one or two antibodies are detected. All conditions indicate a significant small value of negative cells detected, indicating low molecular cross-talk. FIG. 20 and Table 1 shows the results of the contingency table, in which the values for both particle configurations and sizes were higher than 85%, demonstrating that heterofunctional particles allow to identify cells that secrete a specific antibody in a mixture of hybridoma cells with high sensitivity, specificity, and accuracy. Janus and mixed particles with a diameter of 4 μm display higher results.

TABLE 1
Contingency table of different particles sizes and materials for the identification
of a specific hybridoma in a mixture of cells: The results include the sensitivity,
selectivity, and accuracy for the two configurations and the two sizes of particles
compared. The standard error is presented for each configuration.
Mixed Particles

| 2 μm | | | 4 μm | | |
|---|---|---|---|---|---|
| Sensitivity | Specificity | Accuracy | Sensitivity | Specificity | Accuracy |
| 87% ± 6.9% | 90.7% ± 6.0% | 89.2% ± 6.2% | 96% ± 7.4% | 98.2% ± 2.4% | 97.2% ± 5.2% |

Janus Particles

| 2 μm | | | 4 μm | | |
|---|---|---|---|---|---|
| Sensitivity | Specificity | Accuracy | Sensitivity | Specificity | Accuracy |
| 85.7% ± 7.4% | 85% ± 6.5% | 85.3% ± 4.3% | 94.1% ± 3.4% | 94.7% ± 6.4% | 94.5% ± 2.6% |

### Example 24. Isolation of Antibody Secreting Cells Specific to H1 Influenza Virus

H1HA-specific antibody secreting cells were isolated using the mixed particles following the procedure illustrated in FIG. 6. Table 2 shows the number of cells collected in each step for day 5 and day 6 post vaccination. The percentage of cells making H3HA-specific antibodies from the groups sorted as H1HA+ and H1HA− was 12% and 27%, respectively (FIGS. 21A and 21B). The percentage of cells making H1HA specific antibodies from the groups sorted as H1HA+ (true positives) and H1HA− (false negatives) was 49% and 33%, respectively as shown in Table 3.

TABLE 2
Number of cells collected in each step post vaccination.

| Post TIV | PBMC | B cells after enrichment | Targeted ASCs | H1 specific ASCs |
|---|---|---|---|---|
| Day 5 | 10,000,000 | 400,000 | 20,000 | 25 |
| Day 6 | 40,000,000 | 2,440,000 | 220,000 | 56 |

TABLE 3
Contingency table for the identification of HI IV
antibody secreting cells from vaccinated patients.

| | | | | | True | | | |
|---|---|---|---|---|---|---|---|---|
| | True | False | True | False | Positives | | | |
| 3 | Positives | Positive | Negatives | Negatives | (%) | Sensitivity | Specificity | Accuracy |
| Day 5 | 15 | 10 | 3 | 2 | 57% | 81.25% | 17% | 53.57% |
| Day 6 | 23 | 33 | 11 | 4 | 41% | 85.19% | 25% | 47.89% |
| | | | | Average | 49% | 83% | 21% | 51% |

The day 6 post-vaccination sample was separately analyzed by ELISpot and had 77 H1HA-specific ASCs and 89 H3HA-specific ASCs from 500,000 PBMC as shown in FIG. 22. Thus, the frequency of H1HA specific ASC was 0.0154% among PBMC, 0.25% among B cells (Table 4), and 2.8% among total ASC (Table 5). Implementing the present platform, 56 H1HA-specific ASC were sorted, where 23 were true positives (41%). This represents a 164-fold enrichment over total B cells with the proposed platform in comparison to the percentage of H1 specific ASCs measured by ELISpot, and 14.7-fold enrichment over total ASC.

TABLE 4
Frequency of H1HA specific ASC among total B cells.

| PBMC | | |
|---|---|---|
| analyzed | B cell frequency per | H1HA specific ASC |
| by ELISpot | 500,000 PBMC* | among total B cells (%) |
| 500,000 | $\frac{500 , 000 \times 2 , 440 , 000}{40 , 000 , 000} = 30 , 500$ | $\frac{77}{30 , 500} \times 100 = 0.25$ |

*The value is extrapolated from the frequency determined by day 6 post QIV,

TABLE 5
Frequency of H1HA specific ASC among total ASC.

| PBMC analyzed | ASC frequency per | H1HA specific ASC |
|---|---|---|
| by ELISpot | 500,000 PBMC* | among total ASC (%) |
| 500,000 | $\frac{500 , 000 \times 220 , 000}{40 , 000 , 000} = 2 , 750$ | $\frac{77}{2 , 750} \times 100 = 2.8$ |

*The value is extrapolated from the frequency determined by day 6 post QIV.

After correlating the intensity of the detection antigen and the concentration of the secreted antibody at a single cell level, the sensitivity, specificity, and accuracy of the platform was analyzed. It was determined that mixed particles allowed identification of specific antibody secreting cells in a mixture of cells.

### Example 25. Materials

Silica microspheres with diameters of 2 μm and 4 μm were purchased from Bangs Laboratories (Fishers, IN). Coupling reagents, including (3-Aminopropyl)triethoxysilane (APTES), glutaraldehyde, acetone, phosphate buffered saline (PBS), RPMI-1630 cell media were purchased from Sigma-Aldrich (St. Louis, MO), low glucose DMEM from Gibco (11885-084), fetal bovine serum (FBS) from Atlanta Biologicals (Atlanta, GA). Bovine serum albumin (BSA) were purchased from Sigma-Aldrich (St. Louis, MO). Thiol-poly(ethylene glycol)-biotin was purchased from Nanocs (New York, NY). APC Anti human Anti-CD3, APC Anti human Anti-CD28, APC cy7 Anti human Anti-CD3, APC cy7 Anti human Anti-CD28, APC Anti-mouse/human Anti-CD44, Anti human Anti-IL2, Anti human Anti-VEGF, FITC Anti human Anti-IL2, PE Anti human Anti-VEGF, FITC mouse anti-human CD107a antibody from Biolegend (San Diego, CA), human IgG isotype control and IgG Total Mouse Uncoated ELISA Kit with Plates from Thermofisher (Waltham, MA). Protein G was purchased from Protein Specialists (East Brunswick, NJ). Human IL-2 DuoSet ELISA and Human VEGF DuoSet ELISA were obtained from Fisher/R&D systems (China). Lightning-Link® Streptavidin from Expedeon (San Diego, CA). Jurkat cells (CRL-1990), TIB 147 hybridoma cells, and human bone marrow-derived mesenchymal stem cells were obtained from ATCC (Manassas, VA). Blue cell tracker and concanavalin A Alexa Fluor 488 were purchased from Sigma-Aldrich (St. Louis, MO).

### Example 26. Cell Culture

Jurkat and TIB147 hybridoma cells were cultured using RPMI-1640 and DMEM cell culture media, respectively, both supplemented with 1% penicillin-streptomycin and 10% FBS. Human mesenchymal stem cells (hMSCs) were cultured in 89% low glucose DMEM culture media supplemented with 10% FBS, and 1% antibiotic-antimycotic. Cells were incubated in a humidified incubator at 37° C. with 5% CO₂.

### Example 27. Preparation of Janus Particles

Novel Janus particles of 2 μm and 4 μm in diameter targeting the three cell testbeds were fabricated. Briefly, the gold hemisphere was achieved by evaporating titanium and gold onto a monolayer of particles, which were resuspended and modified via thiol-PEG-biotin selectively bound to streptavidin conjugated with the targeting antibody. In the case of Jurkat cells, both anti-CD3 and anti-CD28 were conjugated in equal amounts, which were functional for targeting as well as cell activation. In the case of TIB147 hybridoma cells, anti-CD44 were conjugated for specific targeting. Finally, hMSCs were targeted using anti-CD44. The collecting molecule was immobilized on the silica hemisphere orthogonally as described by Ramirez et al. See Ramirez, K., Campbell, E., Han, S. Y., Buehler, J., Phan, T., Young Yoon, H., Lee, Y. L., Suresh, T., & Sulchek, T. (2019). Optimization of microparticle reagents to collect and detect antibody. Langmuir, 35(36), 11717-11724. The silica surfaces of the particles were functionalized with 2% APTES solution in acetone for 2 minutes followed by incubation with 10% glutaraldehyde in DI water for 10 minutes. After functionalization and multiple phosphate buffered saline (PBS) washes, and the collecting molecule was incubated for one hour, followed by multiple PBS washes to remove excess protein. After particles were washed with PBS, they were resuspended with 10% bovine serum albumin (BSA) Blocking Buffer for 1 hour and washed with PBS to remove excess BSA. The collecting antibody was conjugated by binding streptavidin using Lightning-Link Streptavidin Conjugation Kit following the manufacturer's instructions and incubated for 1 hour. The targeting antibody bind to the biotinylated surface and particles were then washed with PBS to remove excess of the targeting antibody. In the case of Jurkat cells, anti-IL2 collecting molecule was incubated for 1 hour at room temperature and at a concentration of 5 g/L concentration for 1 hour at room temperature. In the case of TIB147 hybridoma cells, protein G was immobilized as the collecting molecule at the same conditions. Protein G (5 g) was incubated for one hour. In the case of hMSCs, anti-VEGF was covalently bound to the silica surface.

### Example 28. Preparation of Mixed Particles

Mixed functionalized silica particles with diameters of 2 μm and 4 μm were prepared following the protocol developed previously to target the three cell testbeds. Briefly, particles were prepared in batches in numbers to have a total surface area of 474 mm² per sample and functionalized with APTES and glutaraldehyde to covalently bind protein G. A mixture of 4 μg/l containing the targeting and collecting antibodies in equal proportions was incubated for one hour at room temperature to immobilize the antibodies in random locations on the particle surface. In the case of Jurkat cells, anti-CD3 and anti-CD28 were added as the targeting molecule and anti-IL2 as the collecting molecule conjugated in equal amounts. In the case of TIB147 hybridoma cells, 2 μg/l anti-CD44 were conjugated for specific targeting, to allow sufficient targeting antibody binding while avoiding steric hindrance of the secreted antibody collection by protein G. Finally, for hMSCs anti-CD44 was conjugated as the targeting molecule and anti-VEGF as the collecting molecule. Particles were then washed with PBS and resuspended with 10% BSA Blocking Buffer for 1 hour and washed with PBS to remove excess of BSA.

### Example 29. Evaluation of Jurkat Cells Activation

Jurkat cell activation was measured by fluorescently staining for CD107a, expressed on the surface of activated lymphocytes. Jurkat cells were activated by incubating 1×10⁵ cells with Janus and mixed particles in a ratio of 1:100 cell: particles for 1 hour. Janus and mixed particles displaying both anti-CD3 and anti-CD28 were used and were also compared to the Dynabead activation (ThermoFisher) as a positive control. Following the manufacturer's instructions, 1×10⁵ cells in 100 μL of RPMI cell media were incubated with 10 ng/ml phorbol myristate acetate (PMA) and 2.5 μM Ionomycin for 4 hours. After incubation with the different activation particles, cells were washed twice with cold RPMI media and incubated with 5 μg/L of FITC anti-CD107a for 20 minutes on ice. Cells were then analyzed using flow cytometry to measure mean fluorescent intensity (MFI) and the signal to noise ratio (SNR), defined as the MFI of the sample/MFI of the negative control of cells with no activating step. Student t-tests were used to compare the different activation techniques and a control of non-activated cells to determine the significance of each approach. The intensity of anti-CD107a was measured using a Zeiss laser scanning confocal microscope (Zeiss LSM 510 VIS Confocal Microscope). The MFI was determined for single cells using the ZEN 2.3 SP1 (Zeiss) software, where the particles were excluded and the cell surface intensity was analyzed.

### Example 30. Evaluation of Cell Viability after Activation

The viability of activated cells during three consecutive days were measured for each experimental condition. Activated cells were incubated in a 96-well plate (Corning; Corning, NY) with 100 μl of fresh cell media in a humidified incubator at 37° C. with 5% CO₂ for one, two, and three days. After the incubation time, the percentage of viable cells via trypan blue exclusion was calculated by dividing the number of viable cells over the total number of cells. Multi-variable ANOVA was used to determine the significance of the percentage of viable cells for each condition.

### Example 31. Evaluating Cell Targeting for Janus and Mixed Particles of Different Sizes

Janus and mixed particles of 2 μm and 4 μm particles were compared to investigate the capability to target cells and to evaluate the optimal number of initial particles per cell. Particles functionalized with anti-CD3 APC and anti-CD28 APC were combined with Jurkat cells in a ratio of 1:100 particles and incubated the sample in a 25 mm glass bottom dish. The number of targeted and untargeted cells was determined via confocal microscopy and manual counting by evaluating all the cells contained in the glass dish. The percentage of cells targeted was calculated by dividing the number of cells with attached particles by the total number of cells in the sample. The number of particles per cell after the FACS isolation step was also determined. For this experiment, cells and particles were combined and cells targeted via FACS were sorted by gating cells marked with blue cell tracker, representing viable cells, and APC, representing the particles labeled with APC anti-CD3 and APC anti-CD28. Cells were then transferred to a glass bottom dish to count the number of particles per cell via confocal microscopy. Multi-variable ANOVA was applied to determine the significance of the number of particles per cell after FACS implementation for each experimental condition of the particle size and particle type.

### Example 32. Comparison Between Janus and Mixed Particles to Isolate High and Low IL2 Secreting Jurkat Cells

The capability of Janus and mixed particles of 2 μm and 4 μm diameter to isolate high and low IL-2 secreting Jurkat cells was evaluated. Jurkat cells were incubated with particles following the protocol described previously. Particles were conjugated with APC anti-CD3 and APC anti-CD28 as the targeting and activating molecules and clear anti IL2 as the collecting molecule as shown in FIG. 7. Briefly, cells and particles were incubated in a ratio of 1:100 at room temperature under gentle agitation for 10 minutes. Cells targeted with particles were sorted using FACS by gating cells marked with blue cell tracker, representing viable cells and APC corresponding to the targeting antibody (anti-CD3 and anti-CD28 in equal amounts) conjugated to the particles. Cells were then incubated in a 96 well plate maintaining a volume and a concentration of 100 μl and 10,000 cells/well. After 2 days of incubation, 5 μg of human IgG and 10% BSA Blocking Buffer was added and cells were incubated for 20 minutes at room temperature to block possible nonspecific binding sites. The secretion rate of the cells was evaluated by adding 5 μg of FITC IL2 antibody for one hour as the detection step and collected cells via FACS with high and low FITC MFI, representing cells with high and low IL-2 secretion, respectively. The gating strategy used is illustrated in FIG. 3C. After cell isolation, the cells were incubated overnight in a 96 well plate maintaining a concentration of 100 μl and 1,000 cells per well. After 24 hours of incubation, supernatants from each well were then analyzed using ELISA specific for IL-2 to validate the secreted concentration of IL-2. After determining IL-2 concentration of each group and for each particle type, multi-variable ANOVA was implemented to evaluate the difference of IL-2 concentration between unsorted, low, and high IL-2 secreting cells for each particle type. The percentage increase of IL2 production from high secreting cells compared to the unsorted cells for each particle type was calculated. The percentage of increase was calculated using the mean IL-2 concentration and dividing the concentration of IL-2 in high secretors over the concentration of IL-2 in unsorted cells.

### Example 33. Comparison Between Janus and Mixed Particles to Isolate Individual High and Low IL-2 Secreting Cells

Single FACS-sorted cells were analyzed for high and low IL-2 secretion. Janus and mixed particles with a diameter of 2 μm were conjugated with APC anti-CD3, APC anti-CD28, and clear anti-IL2. Particles were then incubated with Jurkat cells in a ratio of 1:100, and cells targeted were sorted via FACS. After 2 days of incubation, human IgG and 10% BSA was added for 20 minutes, followed by 5 ag of FITC IL-2 antibody. Next, the cells were sorted via single cells in a 96 well plate that produced high or low IL-2 according to the gating strategy showed in FIG. 3D. Single FACS-sorted cells were then incubated for 10 days and IL-2 secretion was validated using single-cell ELISA specific for IL-2. A t-test was performed to determine the significant difference of IL-2 concentration between unsorted, high, and low IL-2 secreting cells for each particle type.

### Example 34. Comparison of the Detection Molecule Intensity of Single FACS-Sorted Cells Between Janus and Mixed Particles

To compare the detection antibody intensity (FITC IL2 antibody) of single FACS-sorted cells between Janus and mixed particles, the FITC intensity of 60 cells collected was measured using the index sort function of Aria II (FACS DIVA version III) for each particle type. A t-test was performed to determine the significant difference of the detection antibody intensity between Janus and mixed particles. The intensity of FITC IL-2 antibody on particles of single cells isolated as high and low IL-2 secreting cells was also determined using a Zeiss laser scanning confocal microscope (Zeiss LSM 510 VIS Confocal Microscope).

### Example 35. Inheritability of IL-2 Secretion Rates

The IL-2 concentration of isolated cells that secrete high and low IL-2 levels was compared after 24 hours, 7 days, and 1-month post isolation. Janus particles of 2 μm in diameter were combined with Jurkat cells in a 1:100 cell to particle ratio and cells targeted were sorted via FACS. The cells were incubated for 2 days and isolated via FACS high and low IL-2 secreting cells. The cells were then incubated for 24 hours maintaining a concentration of 1,000 cells/well and a volume of 100 μl. The supernatant was separated for IL-2 ELISA analysis and re-cultured the cells for a period of one week and then again for one month. Cell culture media was refreshed every 2 days to maintain good viability. After one week of incubation, cells were incubated in a concentration of 1,000 cells/well for 24 hours and supernatant was analyzed with IL-2 specific ELISA. Cells were then incubated for 3 more weeks, re-activated with 10 ng/ml PMA and 2.5 μM Ionomycin for 4 hours and washed twice with cell media to remove excess of the activation agent. Cells were incubated for 24 hours with a concentration of 1,000 cells/well and analyzed the supernatant with ELISA specific for IL-2. Multi-variable ANOVA were performed to evaluate the significant difference of IL-2 concentration between high and low IL-2 secreting cells and unsorted cells for the different incubation times.

### Example 36. Multiplexed Isolation of Jurkat Cells Based on Both IL-2 and VEGF Secretion Rates

Janus particles that collect interleukin 2 (IL-2) and vascular endothelial growth factor (VEGF) were combined in equal proportions and incubated with Jurkat cells to isolate high and low VEGF and IL-2 secreting cells. Two sets of 2 μm Janus particles that collect IL-2 and VEGF were prepared. For IL2 collection, APC anti-CD3 and APC anti-CD28 were conjugated as the targeting and stimulatory molecule and clear anti IL-2, as the collecting molecule. Similarly, for VEGF collection APC cy7 anti-CD3/28 and clear anti-VEGF were conjugated (FIG. 8). After mixing cells and particles in a 1:100 cell to particle ratio, we sorted via FACS cells stained with blue cell tracker, representing viable cells, that were positive for APC and APC cy7, indicating targeting of both sets of particles to each cell. The collected cells were then incubated for 2 days in culture. To detect protein collected by the particles, human IgG and 10% BSA was added for 20 minutes to block any available binding site, followed by the addition of 5 μg of FITC anti-IL2 and 5 μg PE anti-VEGF for one hour. Cells were then collected via FACS with high and low values of FITC and PE MFI compared to a negative particle control, representing cells with high and low IL-2 secretion and high and low VEGF secretion respectively. The gating strategy is illustrated in FIG. 3D. Cells were incubated in media overnight in a 96 well plate with a concentration of 1,000 cells/well and 200 μL/well. To validate cells secretion, cell supernatant was separated in equal amounts to perform specific ELISA for IL-2 and VEGF. Multiple t-tests were performed to evaluate the significant difference of VEGF and IL-2 concentration between unsorted cells and high and low IL-2 and VEGF secreting cells.

### Example 37. Isolating High and Low Antibody Hybridoma Producers

The ability of Janus particles of 2 μm in diameter to isolate high and low immunoglobulin G (IgG) hybridoma secreting cells was evaluated. TIB 147 hybridoma cells that produce IgG specific for concanavalin A were implemented in this study. Cells were incubated with particles conjugated with APC anti-CD44 and Protein G following the protocol described previously. Targeted cells with particles were sorted via FACS, and then incubated the cells in a 96 well plate maintaining a volume and a concentration of 100 μl media and 10,000 cells per well. After 2 days of incubation, 5 μg of human IgG isotype control and 10% BSA Blocking Buffer was added for 20 minutes followed by the addition of g of FITC concanavalin A for one hour as the detection molecule. Cells were collected via FACS with high and low FITC MFI, representing cells with high and low IgG secretion. After cell isolation, the cells were incubated overnight maintaining a volume and a concentration of 100 μl and 1,000 cells per well. Supernatants were then analyzed using ELISA specific for IgG to validate the secretion levels. Multiple t-tests were implemented to evaluate the significant difference of IgG concentration between unsorted cells, high, and low IgG hybridoma secreting cells.

### Example 38. Determining the Optimal Process Conditions to Maximize the Number of Particles Attached to hMSCs

In order to implement the workflow for adherent cells, a protocol was established to maximize particle targeting when cells are trypsinized from the plate surface. Human mesenchymal stem cells (hMSCs) and 4 μm mixed particles conjugated with CD44 antibody as the targeting molecule were tested. The number of particles per cell that remain attached when we added the particles was determined (1) before trypsinization in the adherent form, (2) during trypsinization in adherent and suspension form, and (3) after trypsinization when cells regained adherence. Two methods of trypsin treatment were tested. First, particles were added to cells in the adherent form immediately after adding the trypsin and while the cells are still adherent. Second, particles were added immediately after the cells were suspended and washed with complete culture media while still in the suspension form. Trypsinization was performed by adding 50 μl of trypsin to each well for 5 minutes, to then add 100 μl of DMEM complete medium, and collect hMSC's in the suspension form by pipette of the liquid. After washing the cells twice in complete medium to ensure the removal of trypsin, the cells were re-plated in a 96 well plate for at least 4 hours to ensure adherence of cells. A 1:100 cell to particle ratio was used to incubate with gently agitation at room temperature and allowed to adhere in a 96 well plate overnight maintaining a concentration of 10,000 cells per well. The number of particles per cell was manually counted using an inverted bright-field microscope (Eclipse Ti, Nikon). Multi-variable ANOVA was implemented to evaluate the significant difference of the number of particles per cell when particles are added before, during, and after trypsinization.

### Example 39. Comparison Between Janus and Mixed Particles to Isolate High and Low VEGF Secreting hMSCs

The ability of Janus and mixed particles to isolate high and low VEGF secreting hMSCs was compared. Particles were conjugated with APC anti-CD44 as the targeting molecule and clear VEGF antibody as the collecting molecule. Janus and mixed particles of 4 μm in diameter after trypsinization of the cells were incubated in a ratio of 1:100 cell per particle in a concentration of 10,000 cells/well. Unbound particles were removed by washing each well twice with PBS, while cells remain attached to the wells. After removing excess of unbound particles, DMEM complete medium was added and the cells were incubated for 4 days. After trypsinizing and collecting cells, 5 μg of human IgG and 10% BSA was added for 20 minutes to block any available binding site and 5 μg of PE VEGF antibody for one hour to label VEGF collected on the particles. Next, cells were collected via FACS with high and low PE intensity, representing high and low VEGF secreting hMSCs. After 4 days of incubation we validated the secretion level via ELISA specific for VEGF and determined the proliferation index in unsorted cells and low and high VEGF secreting cells. A proliferation index was defined as the sum of the number of cells in the second generation after 4 days of incubation and the number of parent cells that were initially obtained from FACS, all divided by the number of parent cells. Multiple t-tests were implemented to evaluate the significant difference of VEGF concentration between unsorted cells, high, and low VEGF secreting cells.

### Example 40. Comparison of the Detection Molecule Intensity in hMSCs

The intensity of PE-anti-VEGF on particles of single hMSCs cells isolated as high and low VEGF secreting cells was determined using a Zeiss laser scanning confocal microscope (Zeiss LSM 510 VIS Confocal Microscope) and using the ZEN 2.3 SP1 (Zeiss) software, where the areas that include only the particles, and the complete area of the cell were analyzed.

### Example 41. Evaluation of Jurkat Cells Activation

The ability of Janus and mixed particles conjugated with anti-CD3 and anti-CD28 stimulating antibodies to bind and activate Jurkat cells was tested. As shown in FIGS. 9A and 9B, both particle types of 2 μm and 4 μm in diameter demonstrated similar activation of Jurkat cells, assessed by CD107a expression compared with common stimulatory protocols such as Ionomycin+PMA and Dynabeads. Similarly, as shown in FIG. 17B, the percentage of viable cells did not decrease, indicating no toxic effect of the particles during a period of 2 days.

### Example 42. Evaluating Cell Targeting for Janus and Mixed Particles of Different Sizes

The capability of both Janus and mixed particles to target Jurkat cells and the number of particles per cell after FACS implementation was analyzed. Janus and mixed particles of 2 μm and 4 μm particles were compared to investigate the capability to target cells and to evaluate the optimal number of initial particles per cell. Particles functionalized with anti-CD3 APC and anti-CD28 APC were combined with Jurkat cells in a ratio of 1:100 particles and incubated the sample in a 25 mm glass bottom dish. The number of targeted and untargeted cells were determined via confocal microscopy and manual counting by evaluating all the cells contained in the glass dish. The percentage of cells targeted was calculated by dividing the number of cells with attached particles by the total number of cells in the sample. The number of particles per cell after the FACS isolation step was also determined. For this experiment, cells and particles were combined and sorted cells targeted via FACS by gating cells marked with blue cell tracker, representing viable cells, and APC, representing the particles labeled with APC anti-CD3 and APC anti-CD28. Cells were then transferred to a glass bottom dish to count the number of particles per cell via confocal microscopy. Multi-variable ANOVA was applied to determine the significance of the number of particles per cell after FACS implementation for each experimental condition of the particle size and particle type.

### Example 43. Comparison Between Janus and Mixed Particles to Isolate High and Low Secreting IL2 Jurkat Cells

The ability of Janus and mixed particles of 2 μm and 4 μm in diameter to isolate high and low secreting IL-2 Jurkat cells was tested. To validate the secretion after sorting, the two groups of sorted cells, as well as unsorted control cells, were analyzed with ELISA specific to IL-2. As shown in FIG. 10A, all groups demonstrated statistical difference between high secreting cells, low secreting cells, and unsorted cells. A greater difference in IL-2 production between the populations was observed using 2 μm Janus particles. Similarly, the highest percentage increase was shown using 2 μm Janus particles, followed by 4 μm Janus particles, 2 μm mixed particles, and 4 μm mixed particles. Low and high secreting cells demonstrated lower and higher IL-2 concentration, respectively. The above is shown in FIG. 10B, where the color emitted by the ELISA results due to IL-2 detection of high secreting cells is darker than unsorted and low secreting cells, from the same figure the color of high secreting cells for 2 μm Janus is darker than for 4 μm Janus particles.

### Example 44. Comparison Between Janus and Mixed Particles to Isolate Individual High and Low IL-2 Secreting Cells

IL-2 secretion of single FACS-sorted cells grouped as high and low IL-2 secreting cells via ELISA specific to IL-2 and the intensity of the detection antibody for mixed and Janus particles was analyzed. After grouping low and high secreting cells as demonstrated in FIGS. 11A through 11C, the concentration of IL-2 validated with ELISA for each group is significantly different for both particle types. Similarly, FIG. 10B shows fluorescent microscope images and their respective detection antibody intensities of single cells isolated via mixed particles and grouped as high or low IL-2 secreting cells, in which high secreting cells demonstrated higher MFI compared with low secreting cells. The detection antibody intensity of single cells for both type of particles was also compared. As shown in FIG. 11C, the intensity generated from the detection antibody is significantly higher for Janus particles compared with mixed particles.

### Example 45. Inheritability of IL-2 Secretion Rates

The sustainability of IL-2 secretion levels on activated Jurkat cells was analyzed over time periods of 24 hours, one week, and one month. As shown in FIGS. 12A and 12B, the secretion rates of isolated high and low IL-2 secreting cells was not significant different after a period of one week and one month.

### Example 46. Isolation of Jurkat Cells Based Upon Secretion of Both IL-2 and VEGF Secretion Rates

The possibility to isolate cells with different secretion levels of two proteins was evaluated, in this case IL-2 and VEGF (FIG. 8). After activating Jurkat cells and incubating targeted cells with VEGF and IL-2 collecting particles, four groups of cells were sorted as indicated in FIG. 3D. Cell secretion levels were evaluated using ELISA specific for IL-2 and VEGF. Although the difference is not statistically different, it can be appreciated in FIG. 13 that low and high secreting cells demonstrated lower and higher concentrations of both IL-2 and VEGF, respectively.

### Example 47. Isolation of High and Low IgG Hybridoma Secreting Cells

The ability of the platform to isolate high and low IgG hybridoma producer cells was also demonstrated by combining 2 μm Janus particles with hybridoma cells and isolated high and low IgG producers via FACS. As is illustrated in FIG. 14, the validation of secretion levels via ELISA demonstrates statistical difference of IgG concentration between high secreting cells, low secreting cells, and unsorted cells, showing higher IgG concentration in high secreting cells.

### Example 48. Comparison Between Janus and Mixed Particles to Isolate High and Low VEGF Secreting hMSCs

To implement our platform in adherent cells a new protocol was developed to minimize particle detachment during trypsinization. The number of particles per cell was determined before, during (in adherent and suspension form), and after trypsinization. As shown in FIGS. 14B and 14C, a significantly higher number of particles per cell after trypsinization is demonstrated compared to when particles were added before or during cell trypsinization. Therefore, the cells were first trypsinized and allowed to adhere, before adding the particles and washing the sample to remove unbound particles to finally incubate the cells during 4 days for VEGF secretion and collection. High and low VEGF secreting cells were sorted via FACS and validated the secretion using ELISA specific for VEGF. FIGS. 15A through 15D illustrate a significant difference between low and high VEGF secreting cells with a significantly higher difference using Janus particles; however, after the second passage, the VEGF secretion rate of the isolated and unsorted cells did not show any difference (FIGS. 15C and 15D). The intensity of the PE antibody in cells isolated for high and low VEGF secretion was compared via confocal microscopy. The MFI of high secreting cells is higher than low secreting cells.

The number of particles per cell that remain attached when particles were added before trypsinization in the adherent form, during trypsinization in adherent and suspension form, and after trypsinization when cells regained adherence were determined.

### Example 49. Proliferation Index of High Secreting Cells

The proliferation index of isolated high and low VEGF secreting hMCSs was evaluated and determined that the proliferation index of isolated high secreting cells implementing both particle types is significantly higher than unsorted and low secreting cells immediately after FACS sorting (FIGS. 16A and 16B). However, the proliferation index did not demonstrate any significant difference between the different groups implementing both types of particles once the cells are passaged the second time (FIG. 16C).

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### LIST OF EMBODIMENTS

Embodiment 1: A composition comprising: a cell capable of secreting one or more molecules, the cell non-covalently attached to a particle, wherein the particle comprises a first linker linking a first unit, the first unit capable of binding to the one or more molecules secreted by the cell, optionally, wherein the one or more molecules secreted by the cell are bound to the first unit.
Embodiment 2: The composition of embodiment 1, wherein the cell is non-covalently bound to the particle through a second unit affixed to the particle via a second linker.
Embodiment 3: The composition of any preceding embodiment, wherein the first linker comprises a silanization binding agent, a carbodiimide binding agent, a carboxylic binding agent, a phosphate binding agent, or combinations thereof.
Embodiment 4: The composition of any preceding embodiment, wherein the second linker comprises a thiol-polymer chain-bioactive molecule complex.
Embodiment 4: The composition of any preceding embodiment, wherein the first unit and second unit each independently comprise a molecule, an antibody, a protein, or combinations thereof.
Embodiment 6: The composition of any preceding embodiment, wherein the first unit comprises a collector molecule and the second unit comprises a targeting molecule.
Embodiment 7: The composition of any preceding embodiment, wherein the first unit comprises a collector antibody and the second unit comprises a targeting antibody.
Embodiment 8: The composition of any preceding embodiment, wherein the first unit comprises a collector protein and the second unit comprises a targeting protein.
Embodiment 9: The composition of any preceding embodiment, wherein the second unit is configured to non-covalently attach to a specific cell.
Embodiment 10: The composition of any preceding embodiment, wherein the first unit is configured to bind to the one or more molecules secreted by the specific cell.
Embodiment 11: The composition of any preceding embodiment, wherein the composition is configured for use to detect the one or more molecules secreted from the cell.
Embodiment 12: The composition of any preceding embodiment, wherein the composition is configured for use to capture the one or more molecules secreted from the cell.
Embodiment 13: The composition of any preceding embodiment, wherein the composition is configured for use to quantify the one or more molecules secreted from the cell.
Embodiment 14: The composition of any preceding embodiment, wherein the composition is configured for use to isolate the cell through fluorescence-activated cell sorting (FACS).
Embodiment 15: The composition of any preceding embodiment, wherein the particle further comprises an outer surface comprising one of hydroxyl or carboxyl functional groups such that the first linker is capable of covalently bonding with the outer surface of the particle.
Embodiment 16: The composition of any preceding embodiment, wherein the particle further comprises a coating comprising metallic functional groups capable of bonding with the second linker, wherein the coating is positioned on at least a portion of the outer surface of the particle.
Embodiment 17: The composition of any preceding embodiment, wherein the coating comprises a pattern such that the first unit and the second unit are arranged along the particle in a pattern.
Embodiment 18: The composition of any preceding embodiment, wherein the coating is positioned on approximately half of the outer surface of the particle, such that a first half of the particle comprises hydroxyl functional groups and a second half of the particle comprises metallic functional groups.
Embodiment 19: The composition of any preceding embodiment, wherein the first half of the particle comprises the first unit and the second half of the particle comprises the second unit.
Embodiment 20: The composition of any preceding embodiment, wherein the particle comprises a diameter ranging from about 0.01 μm to about 100 μm.
Embodiment 21: A composition comprising: a particle comprising a cell-binding unit and a molecule-collection unit, wherein the particle is configured to bind to a specific cell and collect one or more secreted molecules from the specific cell; and optionally, wherein the particle is bound to the specific cell and the one or more secreted molecules.
Embodiment 22: The composition of any preceding embodiment, wherein the molecule-collection unit is bound to the particle via a first linker.
Embodiment 23: The composition of any preceding embodiment, wherein the cell-binding unit is bound to the particle via a second linker.
Embodiment 24: The composition of any preceding embodiment, wherein the first linker comprises a silanization binding agent, a carboxylic binding agent, a phosphate binding agent, or combinations thereof.
Embodiment 25: The composition of any preceding embodiment, wherein the second linker comprises a thiol-PEG-biotin complex.
Embodiment 26: The composition of any preceding embodiment, wherein the molecule-collection unit and the cell-binding unit each independently comprise a molecule, an antibody, a protein, or combinations thereof.
Embodiment 27: The composition of any preceding embodiment, wherein the composition is further configured to detect the one or more molecules secreted from the specific cell.
Embodiment 28: The composition of any preceding embodiment, wherein the composition is further configured to capture the one or more molecules secreted from the specific cell.
Embodiment 29: The composition of any preceding embodiment, wherein the composition is further configured to quantify the one or more molecules secreted from the specific cell.
Embodiment 30: The composition of any preceding embodiment, wherein the particle further comprises an outer surface comprising one of a hydroxyl or a carboxyl functional groups such that the first linker is capable of covalently bonding with the outer surface of the particle.
Embodiment 31: The composition of any preceding embodiment, wherein the particle further comprises a coating comprising metallic functional groups capable of bonding with the second linker, wherein the coating is positioned on at least a portion of the outer surface of the particle.
Embodiment 32: The composition of any preceding embodiment, wherein the coating is positioned on approximately half of the outer surface of the particle, such that a first half of the particle comprises hydroxyl functional groups and a second half of the particle comprises metallic functional groups.
Embodiment 33: A method of isolating and expanding a cell, the method comprising: contacting the cell with a particle, the particle comprising a cell-binding unit and a molecule-collection unit; binding the cell via the cell-binding unit of the particle; capturing one or more molecules secreted from the cell via the molecule-collection unit of the particle; and sorting the cell bound to the particle, optionally by one or more detecting units specific to the one or more molecules secreted from the cell.
Embodiment 34: The method of any preceding embodiment, further comprising releasing the cell from the particle.
Embodiment 35: The method of any preceding embodiment, further comprising expanding the cell.
Embodiment 36: The method of any preceding embodiment, further comprising detecting a quantity of the one or more molecules secreted from the cell, optionally using the one or more detecting units specific to the one or more molecules secreted from the cell.
Embodiment 37: The method of any preceding embodiment, further comprising separating a low-secretion cell from a high-secretion cell based on the quantity of the one or more molecules secreted from the respective cell.
Embodiment 38: A method of isolating and expanding a cell, the method comprising: contacting the cell with a first particle and a second particle, each respective particle comprising a cell-binding unit and a molecule-collection unit; wherein the cell-binding unit and the molecule-collection unit on the respective particle comprise at least one of a molecule, an antibody, or a protein; and wherein the cell-binding unit and the molecule-collection unit on the first particle are different than the cell-binding unit and the molecule-collection unit on the second particle; binding the cell at a first position via the cell-binding unit of the first particle; binding the cell at a second position via the cell-binding unit of the second particle; capturing one or more first secreted molecules from the cell via the molecule-collection unit of the first particle; capturing one or more second secreted molecules from the cell via the molecule-collection unit of the second particle; sorting, by one or more first detecting units specific to the one or more first secreted molecules from the cell based on a type of first secreted molecule; and sorting, by one or more second detecting units specific to the one or more second secreted molecules from the cell based on a type of second secreted molecule.
Embodiment 39: The method of any preceding embodiment, further comprising sorting the cell based on a quantity of the first secreted molecule and second secreted molecule from the cell using the one or more first detecting units and one or more second detecting units.
Embodiment 40: The method of any preceding embodiment, further comprising releasing the cell from the first and second particles.
Embodiment 41: The method of any preceding embodiment, further comprising expanding the cell.
Embodiment 42: A method of producing secreted antibodies, the method comprising: contacting a cell with a particle, the particle comprising a cell-binding unit and a molecule-collection unit; capturing one or more secreted molecules from the cell via the molecule-collection unit of the particle; and sorting the cell bound to the particle, optionally by one or more detecting units specific to the one or more molecules secreted from the cell.
Embodiment 43: The method of any preceding embodiment, further comprising identifying the cell based on a type of the one or more secreted molecules.
Embodiment 44: The method of any preceding embodiment, further comprising isolating the cell based on the type of the one or more secreted molecules.
Embodiment 45: The method of any preceding embodiment, further comprising expanding the cell based on the type of the one or more secreted molecules, such that the cell is capable of producing a specific secreted antibody.
Embodiment 46: The method of any preceding embodiment, further comprising identifying the cell as a high secretion cell or a low secretion cell based on a quantity of the one or more secreted molecules.
Embodiment 47: The method of any preceding embodiment, further comprising simultaneously identifying the cell based on a marker on the cell bound to the particle via the molecule-collection unit of the particle.
Embodiment 48: The method of any preceding embodiment, further comprising isolating the cell based on the quantity of the one or more secreted molecules.
Embodiment 49: The method of any preceding embodiment, further comprising expanding the high secretion cell, such that the high secretion cell is capable of producing a specific secreted antibody.
Embodiment 50: The method of any preceding embodiment, further comprising expanding the low secretion cell, such that the low secretion cell is capable of producing a specific secreted antibody.
Embodiment 51: The method of any preceding embodiment, further comprising generating monoclonal antibodies from the one or more secreted molecules from the cell.

1. A composition comprising:
a particle comprising a first unit;
wherein the first unit is configured to bind to one or more molecules secreted by a cell to which the particle is configured to be bound.

2. The composition of claim 1 further comprising:
the cell;
wherein the first unit is bound to the particle via a first linker; and
wherein the cell is non-covalently bound to the particle through a second unit affixed to the particle via a second linker.

3. The composition of claim 1, wherein the first unit is bound to the particle via a first linker comprising an agent selected from the group consisting of a silanization binding agent, a carbodiimide binding agent, a carboxylic binding agent, a phosphate binding agent, and combinations thereof.

4. The composition of claim 2, wherein the second linker comprises a thiol-polymer chain-bioactive molecule complex.

5. (canceled)

6. The composition of claim 2, wherein at least one of:
the first unit comprises a collector molecule and the second unit comprises a targeting molecule;
the first unit comprises a collector antibody and the second unit comprises a targeting antibody; or
the first unit comprises a collector protein and the second unit comprises a targeting protein.

7.-10. (canceled)

11. The composition of claim 1, wherein the composition is configured for a use selected from the group consisting of detecting one or more of the molecules secreted from the cell, capturing one or more of the molecules secreted from the cell, quantifying one or more of the molecules secreted from the cell, isolating the cell through fluorescence-activated cell sorting (FACS), and a combination thereof.

12.-14. (canceled)

15. The composition of claim 1, wherein the first unit is bound to the particle via a first linker; and
wherein the particle further comprises an outer surface comprising one of hydroxyl or carboxyl functional groups such that the first linker is capable of covalently bonding with the outer surface of the particle.

16. The composition of claim 2, wherein the particle further comprises a coating comprising metallic functional groups capable of bonding with the second linker; and
wherein the coating is positioned on at least a portion of an outer surface of the particle.

17. The composition of claim 2, wherein the particle further comprises:
an outer surface comprising one of hydroxyl or carboxyl functional groups such that the first linker is capable of covalently bonding with the outer surface of the particle; and
a coating comprising metallic functional groups capable of bonding with the second linker;
wherein the coating is positioned on at least a portion of the outer surface of the particle; and
wherein the coating comprises a pattern such that the first unit and the second unit are arranged along the particle in a pattern.

18. The composition of claim 2, wherein the particle further comprises:
an outer surface comprising one of hydroxyl or carboxyl functional groups such that the first linker is capable of covalently bonding with the outer surface of the particle; and
a coating comprising metallic functional groups capable of bonding with the second linker;
wherein the coating is positioned on at least a portion of the outer surface of the particle; and
wherein the coating is positioned on approximately half of the outer surface of the particle, such that a first half of the particle comprises hydroxyl functional groups and a second half of the particle comprises metallic functional groups.

19. The composition of claim 18, wherein the first half of the particle comprises the first unit and the second half of the particle comprises the second unit.

20. The composition of claim 1, wherein the particle comprises a diameter ranging from about 0.01 μm to about 100 μm.

21. The composition of claim 1, wherein the particle further comprises a cell-binding unit;
wherein the first unit is a molecule-collection unit; and
wherein the particle is further configured to;
bind to the cell, being a specific cell, and;
collect one or more of the secreted molecules from the specific cell.

22. The composition of claim 21, wherein the particle is bound to the specific cell and one or more of the secreted molecules; and
wherein the molecule-collection unit is bound to the particle via a first linker.

23. The composition of claim 22, wherein the cell-binding unit is bound to the particle via a second linker.

24. The composition of claim 22, wherein the first linker comprises a silanization binding agent, a carboxylic binding agent, a phosphate binding agent, or combinations thereof.

25. The composition of claim 23, wherein the second linker comprises a thiol-PEG-biotin complex.

26. The composition of claim 21, wherein the molecule-collection unit and the cell-binding unit each independently comprise a molecule, an antibody, a protein, or combinations thereof.

27.-32. (canceled)

33. A method comprising:
contacting a cell with the composition of claim 1, wherein:
the particle further comprises a cell-binding unit; and
the first unit is a molecule-collection unit;
binding the cell via the cell-binding unit of the particle;
capturing one or more molecules secreted from the cell via the molecule-collection unit of the particle; and
sorting the cell bound to the particle.

34. The method of claim 33 further comprising releasing the cell from the particle;
wherein the sorting is by one or more detecting units specific to one or more of the molecules secreted from the cell.

35. The method of claim 34 further comprising expanding the cell.

36. The method of claim 33 further comprising:
detecting a quantity of the molecules secreted from the cell using one or more detecting units specific to one or more of the molecules secreted from the cell; and
separating a low-secretion cell from a high-secretion cell based on the quantity of the molecules secreted from the cell.

37. (canceled)

38. The method of claim 33, wherein:
the contacting comprises:
contacting the cell with a first composition of claim 1 and;
contacting the cell with a second composition of claim 1;
each respective composition comprising a respective particle, each respective particle further comprising the cell-binding unit, and the first unit of each respective particle is a molecule-collection unit;
the cell-binding unit and the molecule-collection unit on the respective particle comprise at least one of a molecule, an antibody, or a protein;
the cell-binding unit and the molecule-collection unit on the first particle are different than the cell-binding unit and the molecule-collection unit on the second particle;
the binding comprises:
binding the cell at a first position via the cell-binding unit of the first particle; and
binding the cell at a second position via the cell-binding unit of the second particle;
the capturing comprises:
capturing one or more first secreted molecules from the cell via the molecule-collection unit of the first particle; and
capturing one or more second secreted molecules from the cell via the molecule-collection unit of the second particle; and
the sorting comprises sorting, by one or more:
first detecting units specific to one or more of the first secreted molecules from the cell based on a type of first secreted molecule; and
second detecting units specific to one or more of the second secreted molecules from the cell based on a type of second secreted molecule.

39. The method of claim 38 further comprising sorting the cell based on a quantity of the first secreted molecule and second secreted molecule from the cell using one or more of the first detecting units and one or more of the second detecting units.

40. The method of claim 38 further comprising releasing the cell from the first and second particles.

41. The method of claim 38 further comprising expanding the cell.

42. A method of producing secreted antibodies comprising:
contacting a cell with the composition of claim 1, wherein:
the particle further comprises a cell-binding unit; and
the first unit is a molecule-collection unit;
capturing one or more secreted molecules from the cell via the molecule-collection unit of the particle; and
sorting the cell bound to the particle by one or more detecting units specific to one or more of the molecules secreted from the cell.

43. The method of claim 42 further comprising at least one of:
identifying the cell based on a type of one or more of the secreted molecules;
identifying the cell as a high secretion cell or a low secretion cell based on a quantity of secreted molecules;
identifying the cell as a high secretion cell or a low secretion cell based on the quantity of secreted molecules while simultaneously identifying the cell based on a marker on the cell bound to the particle via the molecule-collection unit of the particle;
identifying the cell as a high secretion cell or a low secretion cell based on the quantity of secreted molecules and then expanding the high secretion cell, such that the high secretion cell is capable of producing a specific secreted antibody;
identifying the cell as a high secretion cell or a low secretion cell based on the quantity of secreted molecules and then expanding the low secretion cell, such that the low secretion cell is capable of producing the specific secreted antibody;
isolating the cell based on the type of one or more of the secreted molecules;
isolating the cell based on the quantity of the secreted molecules;
expanding the cell based on the type of one or more of the secreted molecules, such that the cell is capable of producing the specific secreted antibody; or
generating monoclonal antibodies from one or more of the secreted molecules from the cell.

44.-51. (canceled)
